(19) United States
(12) Patent Application Publication
(10) Pub. No.: US 2017/0304374 A1
(43) Pub. Date: Oct. 26, 2017

(54) CAPSULE FOR THE ORAL ADMINISTRATION OF BIOPHARMACEUTICALS

(71) Applicant: Symbiotic Health Inc., Roslyn Heights, NY (US)

(72) Inventors: Gerard Honig, Hoboken, NJ (US); Joaquin Urdinez, New York, NY (US); Chandrabali Ghose, New York, NY (US)

(21) Appl. No.: 15/521,259

(22) PCT Filed: Oct. 23, 2015
(86) PCT No.: PCT/US15/57148

§ 371 Date: Apr. 21, 2017

Related U.S. Application Data
(60) Provisional application No. 62/067,839, filed on Oct. 23, 2014.
(60) Provisional application No. 62/145,365, filed on Apr. 9, 2015.

(51) Int. Cl. A61K 35/741; A61K 38/43; A61K 9/48; A61K 39/00; A61K 39/08

(52) U.S. Cl. CPC A61K 35/741; A61K 38/43; A61K 9/4891; A61K 9/48; A61K 39/08; A61K 2039/70

(57) ABSTRACT

In one embodiment the invention provides a capsule for the oral administration of biopharmaceuticals to the gastrointestinal system. The capsule includes a capsule shell enveloping a lipophilic matrix permeated with discrete microcapsules. Each microcapsule is a hydrophilic matrix formed of an internal phase comprising an aqueous medium, stabilized into a discrete structure by a colloidal polymer, and containing the biopharmaceutical(s). The colloidal polymer typically is a hydrocolloid or an amphiphilic colloidal polymer.

### PRIORITY CLAIM

This application claims the benefit of U.S. Provisional Application Ser. No. 62/067,839, filed Oct. 23, 2014, and U.S. Provisional Application Ser. No. 62/145,365 filed Apr. 9, 2015 which applications are hereby incorporated by reference in their entireties.

### TECHNICAL FIELD OF THE INVENTION

This invention relates to the field of biopharmaceutical delivery to the gastrointestinal tract, and more specifically to delivery of probiotic bacteria to the gastrointestinal tract.

### BACKGROUND

Oral delivery of pharmaceutical agents by a capsule is a preferred method of treating diseases in humans or animals. The advantages of a capsule include convenience, comfort and safety of the patient, and the ability to administer multiple doses of a pharmaceutical agent over an extended period of time.

Biopharmaceuticals are an increasingly important class of pharmaceutical agents.

Biopharmaceuticals are generally not compatible with capsules. Biopharmaceuticals, such as proteins or cells, are produced in an aqueous environment and often require the continuous presence of water to retain optimal desired function. Capsules require that the capsule cargo, including the active agent, be dehydrated. Capsule shells are water-soluble to promote delivery of the pharmaceutical agent in the aqueous environment of the gastrointestinal tract. If the capsule is filled with water, the result is an unstable structure that falls apart in a short period of time.

The biopharmaceutical is often dehydrated to prevent it from dissolving the capsule from the inside. However, when a biopharmaceutical is dehydrated the desired physiological function is often not preserved, even when water is reintroduced.

Therefore, there exists a need for a capsule technology capable of delivering biopharmaceutical agents to the gastrointestinal tract.

### SUMMARY

In one embodiment, the invention provides for a therapeutic capsule for the oral administration of a biopharmaceutical to the gastrointestinal system wherein the capsule comprises a capsule shell enveloping a lipophilic matrix permeated with discrete microcapsules, wherein each microcapsule is a hydrophilic matrix formed from an internal phase comprising an aqueous medium, stabilized into a discrete structure by a colloidal polymer. The microcapsules contain a biopharmaceutical.

One embodiment provides for a process for making a therapeutic capsule for the oral administration of a biopharmaceutical to the gastrointestinal system comprising the steps of: a. suspending the biopharmaceutical in an isotonic solution containing a colloidal polymer; b. forming microspheres comprising the colloidal polymer, wherein the polymer forms an internal phase which entraps the complex mixture of biopharmaceutical; c. recovering the microspheres; e. suspending the microspheres in a lipophilic matrix to form a slurry; and, d. packing a capsule with the slurry, wherein the colloidal polymer is selected from the group consisting of hydrocolloid colloidal polymers and amphiphilic colloidal polymers.

One embodiment provides for a therapeutic capsule for the oral administration of bacteria to the gastrointestinal system, comprising a capsule shell enveloping a lipophilic matrix permeated with discrete microcapsules, wherein each microcapsule is a hydrophilic matrix comprising an aqueous medium, stabilized into a discrete structure by a colloidal polymer, and containing the bacteria.

### DETAILED DESCRIPTION

The technologies disclosed herein solve the problem discussed above by providing a therapeutic capsule system for the oral delivery of biopharmaceuticals.

All publications, patent applications, patents, sequences, database entries, and other references mentioned herein are incorporated by reference in their entirety. In case of conflict, the present specification, including definitions, will control.

### Definitions

As used herein, the word “a” or “plurality” before a noun represents one or more of the particular noun. For example, the phrase “a mammalian cell” represents “one or more mammalian cells.”

The term “biopharmaceutical”, which may also be referred to as a biologic medical product or biologic, is any medicinal product manufactured in, extracted from, or semi-synthesized from biological sources. Biopharmaceuticals include vaccines, blood, or blood components, allergenics, somatic cells, gene therapies, tissues, recombinant therapeutic protein, and living cells used in cell therapy. Biopharmaceuticals can be composed of sugars, proteins, or nucleic acids or complex combinations of these substances, or may be living cells or tissues.

They may be isolated from natural sources—human, animal, or microorganism, or produced by means of biological processes involving recombinant DNA technology. Non-limiting examples of the biopharmaceuticals include peptides, carbohydrates, lipids, monoclonal antibodies, biosimilars, biologics, non-IgG antibody-like structures such as but not limited to heterologous antibodies, diabodies, triabodies, and tetrabodies, other multivalent antibodies including scFv2/BITEs, streptabodies, and tandem diabodies, or combinations thereof. Optionally the biopharmaceuticals may be covalently linked to toxins, radioactive materials or another biological molecule, including proteins, peptides, nucleic acids, and carbohydrates. The aforementioned biological molecules include but are not limited to molecules of bacterial origin, viral origin, mammalian origin, or recombinant origin.

The term “chemical pharmaceuticals” includes chemically synthesized pharmaceuticals. Chemical synthesis may refer to a purposeful execution of chemical reactions to obtain a product, or several products.

The term “colony-forming unit” (“CFU”) is a unit used to estimate the number of viable bacteria or fungal cells in a sample, wherein viable is defined as the ability to multiply via binary fission under the controlled conditions.

The term “strain(s) of bacteria” refers to genetic variant or subtype of a microorganism (e.g., virus or bacterium or fungus).

The term “cryoprotectant(s)” in certain instances may refer to a substance used to increase the survival of the cells when frozen and/or thawed, resulting in minimized cell membrane damage due to ice formation. Representative cryoprotectants include glycerol, dimethyl sulfoxide (“DMSO”), ethylene glycol, propylene glycol, butanediol, and polymers such as polyethylene glycol (PEG).

The term “microcapsule” in certain instances may refer to a particle about 0.1 micrometers to 3,000 micrometers in size, which is frequently spherical in shape. The particle may be comprised of a colloid mixture, containing water and a hydrocolloid polymer. The colloid mixture may be a hydrogel. The particle generally has an outer surface separating the interior of the particle from the external environment. In certain instances, the interface may be a shell, comprised of a gel or solid, with physical properties distinct from the interior of the particle.

The term “gastrointestinal tract” in certain instances may refer to the complete system of organs and regions that are involved with ingestion, digestion, and excretion of food and liquids. This system generally consists of, but not limited to, the mouth, esophagus, stomach and or rumen, intestines (both small and large), cecum (plural ceca), fermentation sacs, and the rectum.

The term “colloid” or “colloidal suspension” in certain instances may refer to a mixture of dispersed insoluble particles suspended throughout another substance. Unlike a solution, whose solute and solvent constitute only one phase, a colloid has a dispersed phase and a continuous phase.

The term “complex mixture of bacteria” in certain instances may refer to bacteria of fecal derived bacteria that include multiple strains of bacteria. Often fecal derived bacteria may include at least about 100 to about 1,000,000,000 different strains of bacteria, however the complex mixture of bacteria may refer to about 2 to about 1,000,000,000 strains of bacteria that may require an aqueous environment to maintain their viability.

The term “microbiota” or “microbiome” in certain instances may refer to the set of microorganisms that reside in a particular environment. The “gut microbiota” in certain instances may refer to a set of microorganisms that reside in the gastrointestinal tract, for example the human gastrointestinal tract.

The term “cargo” as used herein refers to the biopharmaceutical or chemical pharmaceutical load delivered by a capsule.

The term “population” refers to all the members of a group. As applied to bacteria it refers to all the organisms of the same strain or species.

The term “synergy”, or “synergistically interact” as applied to bacteria refers to the case where one microorganism helps another to grow or survive. There are examples of a member of the normal microbiota supplying a vitamin or some other growth factor that another microorganism needs in order to grow. This is called cross-feeding between microbes. Another example of synergism occurs during treatment of “staph-protected infections” when a penicillin-resistant staphylococcus that is a component of the normal microbiota shares its drug resistance with pathogens that are otherwise susceptible to penicillin.

### FIGURE LEGENDS

FIG. 1 shows a Schematic Representation of a Stable Capsule.

FIGS. 2a, 2b and 2c show schematic Processes for

Manufacturing a Stable Capsule.

FIG. 3 shows the Scoring System for Capsule Integrity.

FIG. 4 shows the Physical Integrity of Capsule Compositions 45 Minutes After Filling.

FIG. 5 shows the Preservation of anaerobic bacteria in capsules.

FIG. 6 shows the Targeted release of encapsulated polyclonal antibody in simulated gastrointestinal conditions.

### THERAPEUTIC CAPSULE

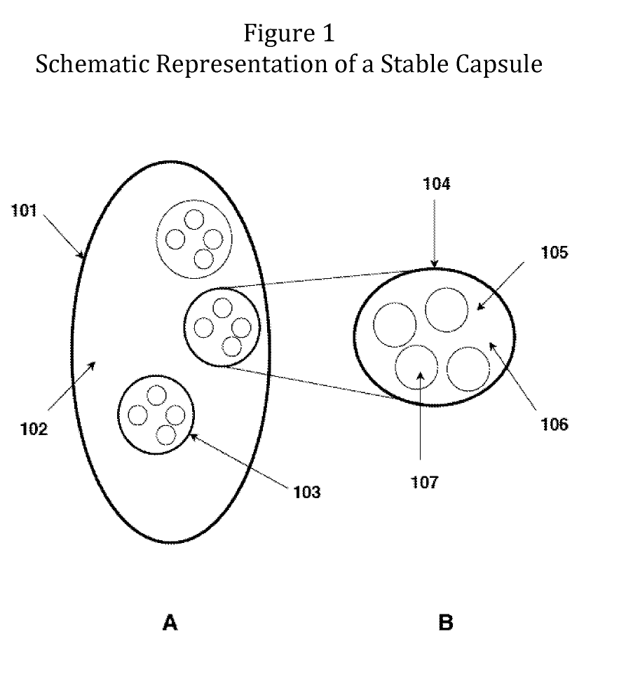

A representative embodiment of the invention is shown in FIG. 1, which schematically depicts a therapeutic capsule that comprises a capsule shell enveloping a lipophilic matrix, wherein the lipophilic matrix is permeated with microcapsules; and the microcapsules entrap a biopharmaceutical agent.

As depicted in FIG. 1, the therapeutic capsule preferably comprises a capsule shell (101) enveloping a lipophilic matrix (102). The capsule shell (101) provides comfortable and safe ingestion, and ensures the targeted release of a biopharmaceutical cargo (107).

The capsule shell may be made of any shell material that is used for the production of capsules used for oral administration of pharmaceuticals. Representative materials used for the manufacture of capsule shells include gelatin or hydroxypropyl methylcellulose (HPMC).

Generally, the capsule shell (101) comprises an acid resistant material that delays the release of the cargo until the capsule reaches the small or large intestine, thereby protecting the cargo from stomach acid and digestive enzymes.

The shell may optionally be coated with a variety of coating materials that confer desirable properties for oral administration, such as acid resistance, time-dependent release, color or taste. Delayed release coatings are well known in the art. Often the coatings are water insoluble at acidic pH of below 3.0, and water soluble at or above about pH 5.5. Exemplarily acid-resist coatings include but are not limited carboxylic group-containing polymers, such as cellulose acetate phthalate (CAP), hydroxypropyl methylcellulose phthalate (HPMCP), hydroxypropyl methylcellulose acetate succinate (HPMC-AS), acrylic copolymers, and shellac. Acid-resistant coatings are well known in the art and are disclosed for example in U.S. Pat. Nos. 4,601,896, 8,710,105, and 8,852,631. Enteric and colon delivery systems are also disclosed in U.S. Pat. Nos. 7,601,347; 7,094,425; 5,407,682; 4,138,013; PCT publication Nos. WO 1995/035100; WO 2013/150331; and EP publication No. 0077956.

Gastrointestinal transit is often simulated in the laboratory. A solution simulating gastric (stomach) fluid is often simulated using a solution with about 0.05 to 0.2 M hydrochloric acid, usually 0.1 M, and with about 0.02% to 1% pepsin enzyme, usually obtained from animal stomach tissue.

Often, the application of delayed release coatings requires a capsule that is physically stable for reasonable periods of time. Generally, capsules filled with aqueous materials are not stable. The inventions disclosed herein provide for capsules that are sufficiently stable for the application of delayed release coatings.

As depicted in FIG. 1, a lipophilic matrix (102) is provided that is permeated with microcapsules (103). FIG. 1 depicts an interface (104) separating the lipophilic matrix and a hydrophilic internal phase of the microcapsule (105). The invention provides that the interface (104) may range from a shell-like outer surface on the microcapsule to a simple border separating the interior of the microcapsule (105) and the lipophilic matrix (102). In some embodiments the interface may be a diffuse border.

The invention provides for an internal phase of the microcapsule (105) comprising a hydrophilic colloid matrix, water, a colloidal polymer (106), and the cargo. The colloidal polymer typically is a hydrocolloid or an amphiphilic colloidal polymer. The hydrophilic matrix may also include physiological salts to maintain the hydrophilic matrix in an isotonic state, and optionally a cryoprotectant to preserve bacteria viability following freezing. Cryoprotectants are well known in the art and include glycerol and PEG 200.

While not being bound by theory, it is believed that the microcapsule is stabilized into a discrete structure by the colloidal polymer(s).

The biopharmaceutical cargo may be discrete structures suspended and immobilized in the colloid matrix, but not dissolved in the matrix (107). These structures may be comprised of bacterial cells, mammalian cells, fungal cells, viruses, proteins, peptides, antibodies, enzymes, small molecules or other structures. Alternatively, a biopharmaceutical cargo maybe comprised of molecules dissolved in the water contained within the colloid matrix of the microcapsule interior (105), such as proteins, peptides, antibodies, enzymes, small molecules or other molecules.

The disclosure provides that the microcapsule may have delayed release properties conferred by the microcapsule material, and/or film coatings applied to the outside of the microcapsule. Such systems are disclosed, for example, in U.S. Pat. No. 8,859,003.

The combination of the microcapsule and the lipophilic matrix has several unexpected benefits. The lipophilic matrix protects the capsule from water on the inner surface of the shell. Often, a capsule shell will degrade and dissolve when there is contact over a sufficient continuous area between the inner surface of the capsule shell and any matrix comprising at or above about 6% volume/volume or more of water. In the absence of the lipophilic matrix, microcapsules in a capsule gradually dehydrate. While not being bound by theory it is believed that the dehydration occurs by capillary action, whereby the capsule shell wicks moisture out of the colloid matrix of the microcapsule into the outer shell, eventually dissolving the water-soluble outer capsule shell, and dehydrating the microcapsule. Conversely, in the absence of the stabilizing action of the colloid matrix, the emulsion of the lipophilic matrix and the hydrophilic matrix is unstable, and eventually separates into distinct phases. Contact of the aqueous phase with the water-soluble capsule dissolves the capsule. The combination of the microencapsulation and the lipophilic matrix unexpectedly confers dramatically improved physical stability and enables the production of industrially useful stable therapeutic capsules.

Typically, the lipophilic matrix includes a digestible oil. It is believed that digestion of the oil in the intestine ensures that the oil will not interfere with the release of the cargo in the GI tract. Representative digestible oils include hydrogenated oil, coconut oil, soybean oil, corn oil, and canola oil.

Optionally, the lipophilic matrix may also comprise a lipophilic cargo released in the GI tract. Exemplary cargo optionally included in the lipophilic matrix includes dehydrated bacteria, dehydrated mammalian cells, proteins, viruses, bacterial spores, small molecules, enzymes, synbiotics, or combinations thereof.

The microcapsules (103) that permeate the lyophilic matrix can generally be characterized as particulate structures comprising a hydrophilic matrix (106) formed from a hydrocolloid or amphiphilic colloidal polymer, and water.

The microcapsules may be comprised of a biopharmaceutical encapsulated in polymer scaffolds for therapeutic delivery. Scaffolds are three-dimensional porous biomaterials that behave as support or confinement structures. They can be non-interactive or interactive, for example promoting targeted delivery of cells or biomolecules.

Scaffolds can be composed of natural, or synthetic biopolymers that form a semi-solid, solid, or hydrogel matrix. Usually, the matrix is a hydrogel, which provides an aqueous environment for the cargo. Usually, the matrix is not highly charged, thus does not negatively affect cell viability or protein folding. The matrix may allow for transport of gases and nutrient to assist in cell survival. For therapeutic cargo delivery, the scaffold should also possess the quality of passive or actively triggered degradation. Often, this degradation is triggered by exposure to a solution containing phosphate buffer at neutral pH.

The synthesis of the scaffold should utilize a crosslinking process, whereby identical molecules are linked into a larger polymer molecule composed of repeating molecular elements. In most embodiments, the crosslinking process does not adversely affect the cargo. For example, in the case of cargo comprising living cells, the crosslinking process is not cytotoxic. Common mechanisms are radical chain polymerization, chemical crosslinking, or a combination of both. The crosslinking process is usually triggered by addition of a hardening agent, and/or a change in the environmental conditions. Preferably, the hardening agent is ionic. More preferably the hardening agent is calcium ions. An environmental change may be a shift in temperature or pH. The ability to trigger crosslinking is often important for the formation of the microcapsules, for example using an emulsion based process; see U.S. Pat. No. 4,822,534A.

Hydrogels are a specific class of polymer scaffolds that are capable of swelling in water or biological fluids, and retaining a large amount of fluids in the swollen state. Their ability to absorb water is due to the presence of hydrophilic groups such as —OH, —CONH—, —CONH2, —COOH, and —SO 3H.

Non-limiting examples of synthetic polymers that may be used for the creation of hydrogels include poly(tetramethylene oxide) (PTMO or PTMEG); poly(dimethyl siloxane) (PDMS); Poly(ethylene glycol) (PEG or PEG); PEG block co-polymers (for example PEG/PD/PDMS, PEG/PPO, PEG/PLGA); poly(lactic-co-glycolic) acid (PLGA); poly(acrylamide) (PAM); hydroxyethyl methacrylate-methyl methacrylate (HEMA-MMA); poly(acrylonitrile)-poly(vinyl chloride) (PAN-PVC); and poly (methylene-co-guanidine) (PMGC).

Non-limiting examples of natural polymers that may be used for the creation of hydrogels include: hyaluronan; chitosan; alginate; alginate complexes (e.g. alginate poly-lysine); gelatin; chondroitin, collagen, elastin, fibrin, xanthan gum, poly-lysine, casein, agarose, alginate, carrageenan, cellulose, gellan gum, guar gum, locust bean gum, pectin, silk fibrin, or combinations thereof.

Preferred hydrocolloid polymers include alginate or pectin. Preferred amphipathic polymers include the protein casein.

Non-limiting examples of hybrid synthetic-natural polymers for the creation of hydrogels include: PEG-fibrinogen; PEG-collagen; PEG-albumin; and pluronic-fibrinogen.

Immobilization of the biopharmaceutical in the microcapsule protects the cargo (107) from the external lipophilic matrix (102).

The use of the hydrocolloid polymer distinguishes the invention from a simple water-in-oil emulsion, in which liquid beads are suspended in a lipophilic matrix. The colloid polymer confers desirable properties, including stabilizing the aqueous microcapsules; prevention of formation of aggregates of aqueous microcapsules, which would clog capsule filling machines; degradation of the capsule shell; prevention of passage of water from the microcapsule into the capsule wall; reduction of shear stress on the bacteria by increasing viscosity of the aqueous phase during mixing; ability to target release of bacteria according to specific conditions which trigger depolymerization and thus release of the cargo from the microcapsule into the gastrointestinal tract; stability of the capsule at different temperatures; production of homogenous products, with microcapsules of specific and stable size, which can easily be manipulated using common capsule filling equipment; and improved preservation of certain types of cargo, especially bacteria and monoclonal antibodies, whose viability in a capsule for oral administration is often improved by microencapsulation.

Moreover, segregation of different cargo within separate microcapsules can be useful if different types of cargo interact negatively during capsule storage.

### Cargo

The cargo (107) generally comprises a biopharmaceutical that requires the continuous presence of water to retain its activity. Representative biopharmaceuticals that may serve as cargo include bacteria, fungi, peptides, viruses, carbohydrates, lipids, and proteins. Preferred biopharmaceuticals include probiotics, synbiotics, bacteria isolated from fecal matter, biosimilars, biologics, polyclonal and monoclonal antibodies, non-IgG like structures such as but not limited to heterologous antibodies, diabodies, triabodies, and tetrabodies, other multivalent antibodies including scFv2/BITEs, streptabodies, and tandem diabodies, or combinations thereof. Non-limiting specific biopharmaceuticals include: LL-37 (cathelicidin), L enantiomer; LL-37 (cathelicidin), D enantiomer; Salmonella typhi Ty21a bacteria; live rotavirus; the antibodies CDA1 and MDX-1388; and beta-lactamase. The aforementioned biological molecules include but are not limited to molecules of bacterial origin, viral origin, mammalian origin, or recombinant molecules.

In some embodiments the cargo is a monoclonal antibody. In some embodiments, the monoclonal antibody may be ramucirumab, vedolizumab, tocilizumab, certolizumab, catumaxomab, panitumumab, natalizumab, bevacizumab, cetuximab, erbitux, adalimumab, basiliximab, infliximab, muromonabCD3, basiliximab, necitumumab or any combination of the above.

In some embodiments the cargo is an enzyme. In some embodiments, the enzyme may be a digestive enzyme or a lactamase.

In some embodiments the cargo is a peptide. In some embodiments, the peptide may be insulin, pramlintide, GLP-1, fenfluramine, somatostatin, interferon, EPO, GM-CSF, polymyxin B, colistin or any combination of the above.

Often the biopharmaceutical may comprise a complex mixture of bacterial species that have been isolated from fecal matter. Typically 1 g sample of fresh fecal matter may have from about 1×10³, to about 1×10⁹ different strains of bacteria. Generally, a 1 g sample of fresh fecal matter often has from about 5×10¹⁰ to about 2×10¹¹ bacterial cells.

Often the cargo includes isolated bacteria, or a mixture of isolated bacteria. Representative bacterial species in the cargo may include Acidaminococcus intestinalis; Bacteroides ovatus; Bifidobacterium adolescentis; Bifidobacterium longum; Clostridium cocleatum; Blautia product; Collinsella aerofaciens; Dorea longicatena; Escherichia coli; Eubacterium desmolans; Eubacterium eligens; Eubacterium limosum; Eubacterium rectale; Eubacterium ventriosum; Faecalibacterium prausnitzii; Lachnospira pectinoshiza; Lactobacillus casei/paracasei; Lactobacillus casei; Ruminococcus torques; Parabacteroides distasonis; Raoultellasp.; Roseburia faecalis; Roseburia intestinalis; Ruminococcus obeum; C. scindens; Barnesiella intestihominis; Pseudoflavonifractor capillosus; and Blautia hansenii; or combinations thereof.

In some embodiments, the therapeutic capsule comprises at least one population of bacteria selected from Table A, or combinations thereof.

TABLE A
Representative Bacterial Cargo

Examples of sugar fermenters
Ethanol fermenters - Saccharomyces
Homolactoc acid fermenters - Lactococcus
Heterolactic acid fermenters - Leuconostoc
Porpionic acid fermenters - Propionobacterium
Mixed acid fermenters - Escherichia
2,3-butanediol fermenters - Enterobacter
Butyrate fermenters - Clostridium
Acetone butanol fermenters - Clostridium
Homoacetic acid fermenters - Acetato bacterium
Examples of spore formers
Clostridium
Bacillus
Sporolactobacillus
Sporosarcina
Examples of non-spore formers
Saccharomyces
Lactococcus
Propionobacterium
Escherichia
Enterobacter
Examples of probiotic strains
Lactobacillus acidophilus
L. fermentum
L. plantarum
L. rhamnosus
L. salivarius
L. gasseri
L. reuteri
Bifidobacterium longum
Bifidobacterium bifidum
Clostridium butyricum

A capsule may have about 5% to about 20% weight/volume of live bacteria. More often a capsule has from about 8% to about 15% weight/volume of live bacteria. Most often the capsule has about 10% to about 12% weight/volume of bacteria.

In certain embodiments a capsule may comprise about 8%, about 9%, about 10%, about 11%, about 12%, about 13%, about 14%, or about 15% weight/volume of bacteria.

In some embodiments a single capsule may contain about 1, about 1×10, about 1×10², about 1×10³, about 1×10⁴, about 1×10⁵, about 1×10⁶, about 1×10⁷, about 1×10⁸, about 1×10⁹, about 1×10¹⁰, about 1×10¹¹, about 1×10¹², about 1×10¹³, about 1×10¹⁴, about 1×10¹⁵, or about 1×10¹⁶ CFUs of bacteria.

The cargo may comprise a homogenous collection, or a heterogeneous assortment of microcapsules. A homogenous collection of microcapsules refers to a group of microcapsule that contains essentially the same cargo. For example a homogenous collection of microcapsules may contain essential the same population of bacteria. In contrast a heterogeneous assortment of microcapsules refers to a group of microcapsules that contain substantially different cargo. For example, a representative heterogeneous assortment of microcapsules includes two different populations of microcapsules, wherein each population of microcapsules comprise a different population of bacteria.

Often different populations of bacteria have different 16S rDNA sequences. 16S rDNA sequencing is a well-known method of classifying bacteria according to operational taxonomic unit (OTU) (see U.S. Pat. No. 6,054,278A). Optionally, different populations of bacteria to be delivered using the capsule described herein have no more than 97% homology between their respective 16S rDNA sequences. Different populations of bacteria may have no more than 90% homology between their respective 16S rDNA sequences. Sometimes different populations of bacteria have no more than 85% homology between their respective 16S rDNA sequences.

In some embodiments the capsule may have 1, 2, 3, 4, 5, 6, 7, 8, 9, 10, or different populations of bacteria isolated from each other by incorporation into microcapsules. The capsule may contain about 1×10, about 1×10², about 1×10³, about 1×10⁴, about 1×10⁵, about 1×10⁶, about 1×10⁷, about 1×10⁸, about 1×10⁹, about 1×10¹⁰, about 1×10¹¹, about 1×10¹², or about 1×10¹³, or more different populations of bacteria isolated from the others by incorporation into microcapsules.

Advantageously, since the therapeutic capsule provides for the separation of populations of bacteria, negative interactions between populations maybe precluded. For example, bacteria of certain strains can secrete enzymes, such as lysins, that perforate the cell membrane of bacteria of other strains. Because the water-soluble lysins do not diffuse through the lipophilic matrix, toxic interactions between different bacterial strains may be precluded.

In an embodiment, a therapeutic capsule is provided that comprises a bacterial composition comprising at least a first type of isolated bacterium, and a second type of isolated bacterium.

In an embodiment, a therapeutic capsule is provided that comprises at least a first type of isolated bacterium and a second type of isolated bacterium, wherein: i) the first type and the second type are independently capable of forming a spore; ii) only one of the first type and the second type are capable of forming a spore or iii) neither the first type nor the second type are capable of forming a spore, wherein the first type and the second type are not identical.

In an embodiment, a therapeutic capsule is provided that comprises at least about 3, 4, 5, 6, 7, 8, 9, or 10 types of isolated populations of bacteria, each isolated population contained in a different population of microcapsules.

In an embodiment, the cargo provides at least about 3, 4, 5, 6, 7, 8, 9, or 10 types of isolated bacteria in one population of microcapsules, and at least 1, 2, 3, 4, 5, 6, 7, 8, 9, or 10 of isolated populations of bacteria in another population of microcapsules.

In an embodiment, a therapeutic capsule is provided that comprises microcapsules that have at least about 5 types of isolated bacteria and at least 2 of the isolated bacteria in each microcapsule.

In some embodiments, a therapeutic capsule is provided that comprises at least about 3, 4, 5, 6, 7, 8, 9, 10, 11, 12, 13, 14, 15, 16, 17, 18, 19, 20, 21, 22, 23, 24, 25, 26, 27, 28, 29, or more different populations of isolated bacteria.

In some embodiments, the therapeutic capsule comprises two different populations of bacteria, each isolated in different microcapsules that are present in approximately equal concentrations. In another embodiment of the capsule, the concentration of a first population is at least about 150% of the concentration of the second type. In another embodiment the concentration of a first population is at least about 150% of the concentration of the second type. In another embodiment the concentration of a first population is at least about 250% of the concentration of the second type. In another embodiment the concentration of a first population is at least about 5 times the concentration of the second type.

In some embodiments, each microcapsule comprises from two to about twenty different populations of bacteria.

In one embodiment, the microcapsules comprises from two to about twenty types of different populations of bacteria, wherein at least two populations of bacteria are not known to be capable of spore formation.

In some embodiments, a first strain of isolated bacterium and a second strain of isolated bacterium are selected from Table 1 of U.S. Pat. No. 8,906,668.

In some embodiments, the microcapsule comprises a first population of bacteria and a second population of bacteria that comprises an operational taxonomic unit (OTU) distinction. In an embodiment, the OTU distinction comprises 16S rDNA sequence similarity below about 95% identity. In an embodiment, the first type of isolated bacterium and the second type of isolated bacterium independently comprise bacteria that comprise 16S rDNA sequence at least 95% identical to 16S rDNA sequence present in a bacterium selected from Table 1 of U.S. Pat. No. No. 8,906,668.

In an embodiment, a first population of bacteria and a second population of bacteria synergistically interact.

In some embodiments, capsules comprise bacteria which are capable of functionally populating the gastrointestinal tract of a human subject to whom the composition is administered.

### Method of Treatment

In another embodiment the invention provides a method of treating a disease in a patient by orally delivering a biopharmaceutical to the gastrointestinal tract with the capsule described herein.

In another embodiment the invention provides a method of treating a disease in a patient by orally delivering bacteria to the gastrointestinal tract with the capsule described herein.

An embodiment provides a method of treating a disease in a patient by administering a stable capsule for the oral administration of a complex mixture of bacteria to the gastrointestinal system.

The patient is generally an animal. Often the patient is a human patient.

The disease may be related to a Clostridium difficile infection of the gastrointestinal system.

Generally, the capsule may be given to a patient on a once daily basis. Often the capsule is administered twice a day.

The treatment period may be determined by one of ordinary skill in the art and will depend on the disease being treated.

Populating the gastrointestinal tract with bacteria and/or biopharmaceuticals may be used to treat diseases. These include preventing and/or treating ailments of: the alimentary tract and metabolism; bile and liver disorders; dysbiosis of the gastrointestinal tract; growth and/or colonization of the gastrointestinal tract by a pathogenic bacterium; reducing growth and/or colonization of the gastrointestinal tract by a pathogenic bacterium; reduction of one or more non-pathogenic bacteria in the gastrointestinal tract; ailments of the alimentary tract and acid-related disorders; nausea; constipation; diarrhea; intestinal inflammation and infections; obesity; diet related disorders; blood and blood forming organs; the cardiovascular system; hypertension or high concentrations of lipid; dermatological systems; genito-urinary system; adrenal pituitary; hypothalamic disorders; pancreatic disorders calcium homeostasis; the immune system; musculoskeletal system and bone disease, musculonervous system diseases, the respiratory system including obstructive airway diseases; cough and cold; other respiratory system diseases; allergies; or combinations thereof.

Representative diseases which may be prevented or treated with the therapeutic capsule include: C. difficile infections of the gastrointestinal system, ulcerative colitis, Crohn's disease, inflammatory bowel disease, irritable bowel disease, colon cancer, appendicitis, allergies, metabolic syndrome, diabetes, liver stenosis, fatty liver disease, kidney stones or combinations thereof.

### Process for Manufacturing the Therapeutic Capsule

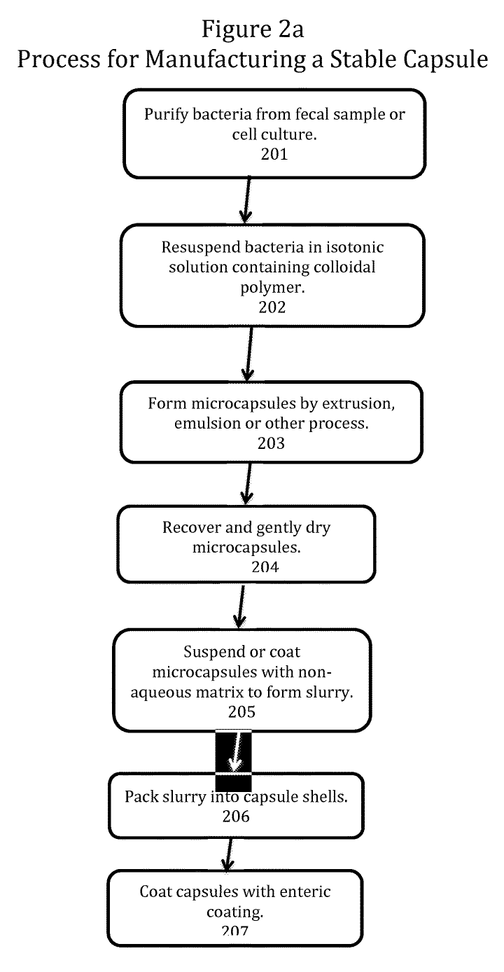

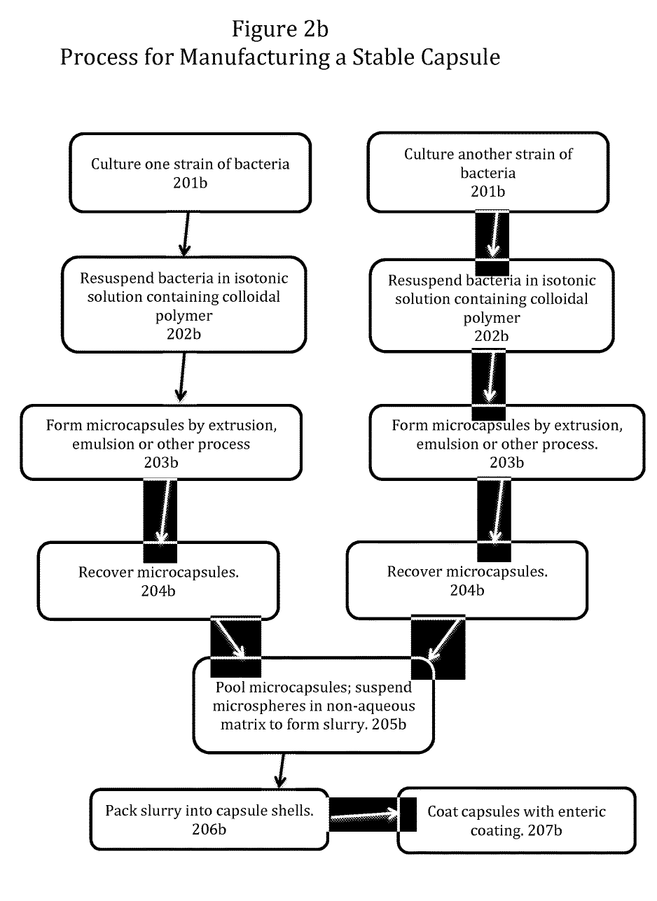

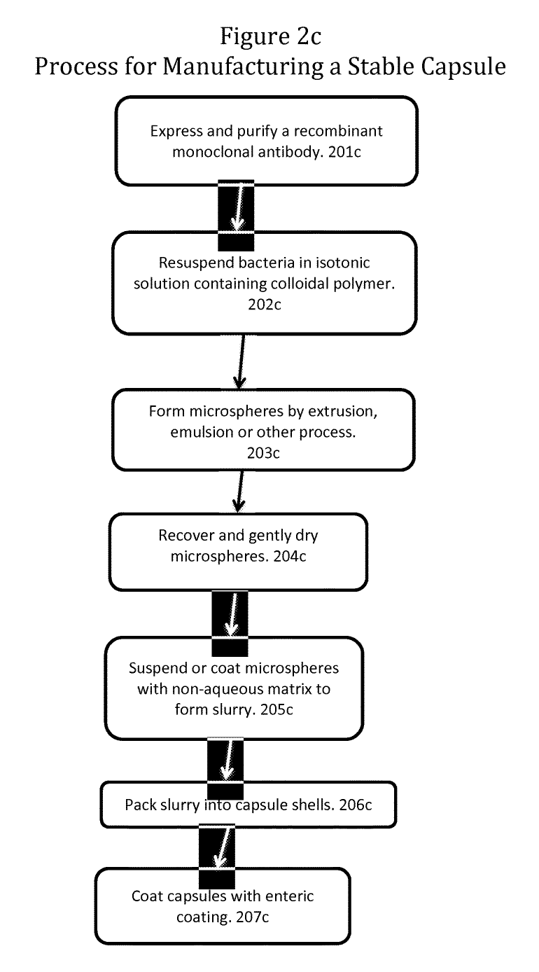

FIGS. 2a, 2b and 2c are flow charts showing the steps in the process of manufacturing the capsule to deliver various cargo.

In one embodiment the first step in this process is the isolation of a complex mixture of bacteria from fecal matter (FIG. 2a) (201). The fecal matter may be harvested from human patients by methods well known in the art. See U.S. Patent Publication No. 2014/0238154. Generally, human fecal samples are collected and immediately chilled on ice. Typically, samples are processed within about 30 minutes to about two hours of collection, usually about one hour of collection. Samples may be slowly homogenized with a sterile isotonic buffer, such as phosphate buffered saline (PBS), about pH 7.0, in a blender. Prior to homogenization the blending chamber is often purged for several minutes with an inert gas, such as nitrogen or argon, to remove oxygen. Blending may be performed with about 40 g to 60 g, often 50 g, of donor feces and about 150 mL to 400 mL, usually 250 mL, of buffer. Typically blended samples are passed through a series of three to five sieves with pore sizes ranging from about 2.0 to about 0.25 mm. The final filtrate passing through the finest sieve may be collected in a conical centrifuge tubes and centrifuged at about 2,000 to about 6,000 rpm, usually about 4,000 rpm for about 10 minutes at about 4° C. The supernatant is generally discarded and the pellet suspended in about one half the original volume of PBS containing 10% glycerol. The mixture of bacteria are typically used immediately, or may be stored frozen at −80° C. See US Patent Publication No. 20140147417.

Generally, the bacteria are suspended in about 500 mL of an isotonic solution containing physiological salts, water-soluble cryoprotectants, and a lyophilic colloidal polymer to form an isotonic suspension (202). Generally, the concentration of the polymer ranges from about 1% to about 4% (w/v), usually about 2% (w/v). The cryoprotectant may be glycerol, or PEG at a concentration from about 8 to about 15% (v/v). The concentration of bacteria is generally about 5% to about 15% weight/volume (w/v), usually about 8% (w/v).

Once suspended in the isotonic solution, the bacteria are entrapped in microcapsules (203). Methods of forming microcapsules containing biopharmaceuticals, such as bacteria, are well known in the art and are described by Chávarri et. al., (2012). Encapsulation Technology to Protect Probiotic Bacteria, Probiotics, Prof. Everlon Rigobelo (Ed.), ISBN: 978-953-51-0776-7, InTech, DOI: 10.5772/50046. Available from: http://www.intechopen.com/books/probiotics/encapsulation-technology-to-protect-probiotic-bacteria. See U.S. Pat. Nos. 5,766,907; 6,242,230; 5,733,568; U.S. Patent Publication No. 2012/0263826; 2006/0099256; Published PCT Applications WO 2015/019307; WO2014/152338.

Microcapsules entrapping bacteria may be formed by an extrusion process, an emulsion process, or other techniques.

Extrusion generally involves filling syringes with an isotonic suspension of bacteria, which is extruded through a hypodermic needle into a hardening solution. The internal diameter of the needle will define the average diameter of the microcapsules. Often extrusion is performed with a 23-gauge needle.

The suspension includes a lyophilic colloidal polymer, which when exposed to the hardening solution, results in the formation of microcapsules encapsulating the complex mixture of bacteria.

The hardening solution frequently includes a hardening agent, such as a divalent cation, that stabilizes the microcapsules. The divalent cation may be Ca²⁺. Generally, the concentration of the divalent cation may be from about 0.05 M to about 0.2 M. Often the concentration is about 0.1 M. Frequently the hardening solution also includes a cryoprotectant such as glycerol, or PEG 200. The cryoprotectant may be at a concentration from about 5 to about 20% w/v. Often the concentration is about 8 to about 17% (v/v). Most often it is about 15% w/v.

Optionally the hardening solution may also include an additional polymer, such a chitosan or polyethylene glycol, to increase the mechanical stability and temperature tolerance of the microcapsules.

The suspension of microcapsules in the hardening solution is generally slowly stirred at room temperature (about 22° C.) for about 5 to about 40 minutes from about 100 to about 300 rpms to allow complete polymerization of the microcapsule. Often the solution is stirred for about 10 to about 30 minutes. Most often the solution is stirred for about 15 minutes.

Emulsification generally involves the mixing of an isotonic suspension of bacteria with an inert oil, such as corn oil or soybean oil. Mixing is generally performed by rapid mechanical agitation from about 1000 to about 2000 rpms. A surfactant, such as Tween 80 at about 0.2% v/v, may be included in the mixture to promote the emulsification. Once a fine emulsion has been generated, a hardening agent is added to the oil phase to polymerize the lyophilic colloidal polymer, and form a suspension of stable microcapsules encapsulating the complex mixture of bacteria.

The microcapsules are stable and may be isolated by filtration or centrifugation (204). Once isolated the microcapsules are generally washed with a solution that includes a divalent cation such as CaCl₂, preferably at concentration of about 0.1 M, to prevent the microcapsules from sticking to each other. After the microcapsules have been isolated the excess water is generally removed by placing the microcapsules on filter paper for about 2 to about 5 minutes.

Microcapsules are generally suspended with a lipophilic matrix to form a slurry (205). The lipophilic matrix is usually a digestible oil such as hydrogenated oil, coconut oil, soybean oil, corn oil or canola oil. Generally about 5 mL of oil may be used for every 5 mL of the microcapsules. The lipophilic matrix prevents direct contact of microcapsules with the inner surface of the capsule shell.

The capsules, such as gelatin or HPMC capsules, are usually filled to about 90% of the capsule volume with the slurry (206).

The capsules are coated with an enteric targeting film, such as a Eudragit polymer (207).

In an embodiment, the microcapsules comprise cultured bacteria. A process for manufacturing such a capsule is illustrated in FIG. 2b, involving: (a) culturing two individual strains of bacteria separately, in appropriate conditions for optimum growth in laboratory (201b); (b) suspending the different cultured bacterial species separately in an isotonic solution containing a lyophilic colloidal polymer (202b); (c) forming microcapsules comprising the hydrophilic colloidal polymer, wherein the polymer forms an internal phase comprising an aqueous medium and bacteria (203b); (d) recovering the microcapsules (204b); (e) suspending the different microcapsules containing different bacteria together in a lipophilic matrix to form a single slurry, preventing dehydration of the microcapsules (205b); (f) packing a capsule with the slurry that represents a mixed population of the different cultured bacteria (206b); and (g) coating the capsule with an enteric targeting film (207b). The capsule includes a capsule shell enveloping a lipophilic matrix permeated with discrete microcapsules. Each microcapsule is a hydrophilic matrix formed of an internal phase comprising an aqueous medium, stabilized into a discrete structure by a colloidal polymer, and containing the bacteria. The colloidal polymer typically is a hydrocolloid or an amphiphilic colloidal polymer.

In an embodiment, the microcapsules comprise a protein cargo. In some embodiments, the protein is an enzyme. In other embodiments, the microcapsules comprise a peptide cargo.

In some embodiments, the cargo is an antibody. A process for manufacturing such a capsule is illustrated in FIG. 2c.

The first step in this process is the expression and purification of the monoclonal antibodies (201c). Full-length monoclonal antibodies are expressed using a recombinant expression system using published cloned DNA sequences.

Generally, the purified monoclonal antibodies are suspended in about 50 mL of an isotonic solution containing a lyophilic colloidal polymer (202c). The process is continued by: forming microcapsules comprising the hydrophilic colloidal polymer, wherein the polymer forms an internal phase comprising an aqueous medium and the complex mixture of bacteria (203c); recovering the microcapsules (204c); suspending the microcapsules in a lipophilic matrix to form a slurry, preventing dehydration of the microcapsules (205c); packing a capsule with the slurry (206c); and coating the capsule with an enteric targeting film (207c). The capsule includes a capsule shell enveloping a lipophilic matrix permeated with discrete microcapsules. Each microcapsule is a hydrophilic matrix formed of an internal phase comprising an aqueous medium, stabilized into a discrete structure by a colloidal polymer, and containing the antibody. The colloidal polymer typically is a hydrocolloid or an amphiphilic colloidal polymer.

In some embodiments, the capsule may be stored prior to use. Storage time may be about 1 day, about 10 days, about 90 days or about 365 days. The storage may occur at a temperature of about 25 degrees Celsius, 4 degrees Celsius, −20 degrees Celsius or −80 degrees Celsius. In some embodiments, the capsules contain bacteria and the starting number of viable bacteria is quantified prior to forming the microcapsules and capsules. In some embodiments, this quantification is performed by CFU estimation. In some embodiments, the number of viable bacteria contained in the capsule is quantified following capsule production and storage. In some embodiments, the number of viable bacteria recovered following capsule production and storage is equal to about 120%, 100%, 99%, 90%, 80%, 70%, 30%, 20%, 5% or 0.2% of the initial amount.

In some embodiments, the capsules contain a protein cargo and the starting amount of functional protein is quantified prior to capsule production and storage. In some embodiments, this quantification is performed by enzyme-linked immunoassay (ELISA). In some embodiments, the amount of functional protein is quantified following capsule production and storage. In some embodiments, the amount of functional protein recovered following capsule production and storage is equal to about 99%, 90%, 80%, 70% or 60% or 30% of the initial amount.

For the disclosure to be better understood, the following examples are set forth. These examples are for purposes of illustration only and are not to be construed as limiting the scope of the disclosure in any manner.

### EXAMPLES

### Example 1

### Manufacturing Capsules with Fecal Matter as the Cargo

Example 1 exemplifies a process for forming a stable capsule for the oral administration of a complex mixture of bacteria to the gastrointestinal system. The process is schematically illustrated in FIG. 2a.

Donor fecal material was immediately chilled on ice. Samples were processed within one hour after collection.

Fecal samples were homogenized by mixing 50 g of donor feces and 250 mL of sterile phosphate buffered saline, pH 7, (PBS) in a Waring Blender Model #700S. The blending chamber was purged with nitrogen gas for several minutes to remove oxygen prior to homogenization. Samples were blended three times with the blender speed set to about 20,000 rpms for 20 seconds. Blended samples were passed through a series of four sieves with pore sizes of 2.0 mm, 1.0 mm, 0.5 mm and 0.25 mm (W.S. Tyler Industrial Group, Mentor, Ohio). The sieves were based on US standard sieve sizes of 10, 18, 35, and 60 for 2.0 mm, 1.0 mm, 0.5 mm and 0.25 mm, respectively. See U.S. Patent Publication No. 20140147417. The 0.25 mm fraction was collected in 50 mL conical centrifuge tubes and centrifuged at 4,000 rpm for 10 minutes at 4° C. The supernatant was discarded and the pellet suspended in one half the original volume of PBS (e.g. 125 mL) containing 15% glycerol. The samples were used immediately.

Microcapsules encapsulating the bacteria were formed by the extrusion method. Syringes were filled with a suspension of 8% weight/volume (w/v) of purified fecal-derived bacteria, 2% w/v sodium alginate and 15% w/v glycerol in PBS, pH=7.0. The suspension was extruded through a 23 gauge needle into 100 mL of 0.1 M CaCl2 solution to form microcapsules encapsulating the bacteria. The solution was stirred for 30 minutes to allow complete polymerization of the microcapsules. The microcapsules were collected with a 70 micron mesh sieve and washed two times with 100 mL of 0.1M CaCl2 to prevent the microcapsules from sticking to each other. The excess solution was removed and the capsules were placed on filter paper for 3 minutes to eliminate the residual external liquid.

Five mL of microcapsules were suspended with 5 mL of hydrogenated oil to form a slurry. The hydrogenated oil prevents direct contact of microcapsules with the inner surface of the capsule shell. Such direct contact would lead to passage of water from the microcapsule into the outer capsule shell by wicking action.

HPMC size 0 capsules were filled to 90% of the capsule volume with slurry of microcapsules in hydrogenated oil.

### Example 2

### Capsule Integrity Assay

Example 2 exemplifies a process for assessing the physical stability of the capsule of the present invention.

To assess capsule integrity HPMC size 0 capsules were filled to 90% volume with one of the following compositions:

a) Aqueous solution made of 15% v/v glycerol and 2% w/v NaCl (labeled as ‘Aqueous matrix alone without microencapsulation’ in Table B);

b) Microcapsules made of 2% w/v Sodium alginate and 15% v/v glycerol (labeled as ‘Microcapsules alone’ in Table B);

c) Microcapsules embedded in hydrogenated oil (5 mL of oil per equivalent of 5 mL of sodium alginate) (labeled as ‘Microcapsules in Lipid Matrix’ in Table B); or

d) NaCl (labeled as ‘Powder’ in Table B); or

e) Aqueous matrix, not microencapsulated, mixed with hydrogenated oil (labeled as ‘Aqueous matrix with lipid matrix without microencapsulation’ in Table B); or,

Hydrogenated oil alone (labeled as ‘Lipid matrix alone’ in Table B).

TABLE B
Proportion of capsules with an intact shell
following storage at room temperature.

| | Initial | 24 hours |
|---|---|---|
| Capsule Design | % Intact | % Intact |
| Microcapsules in | 100% | 100% |
| lipid matrix | | |
| Microcapsules alone | 100% | 0% |
| Aqueous matrix | 100% | 0% |
| alone without | | |
| microencapsulation | | |
| Aqueous matrix, | 100% | 0% |
| without | | |
| microencapsulation, | | |
| with lipid matrix | | |
| Lipid matrix alone | 100% | 100% |
| Powder | 100% | 100% |

Capsules were maintained at RT (about 22° C.) for the duration of the experiment. Capsule integrity was assessed by visual inspection at 0, 30 minutes, 1 hr, 2, hrs, 5 hrs, and 24 hrs.

Visual inspection was used to assign scores based on a capsule's integrity. Capsules were scored as having either an intact or broken/leaking capsule shell at the indicated times.

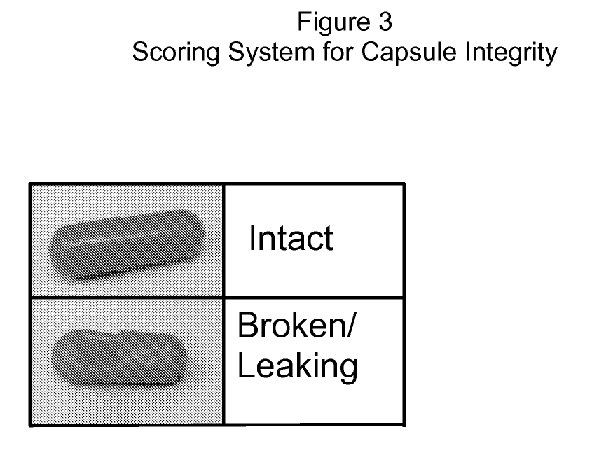

FIG. 3 shows photographs of an intact HPMC size 0 capsule and a broken/leaking capsule.

Table B shows the results of the experiment in which the stability of capsules having one of compositions of (a) to (e) described above. The proportion of capsules with an intact shell at the indicated time was noted for each type of capsule.

Different capsule designs were produced, in order to assess the physical stability. As positive controls, capsules were filled with compositions known to confer long-term capsule stability. These included capsules filled with hydrogenated oil or with a dry powder. These capsule systems are not compatible with retaining a cargo in an aqueous matrix. To produce capsules that might be stable when filled with aqueous cargo, the aqueous matrix, without microencapsulation, was mixed with a lipid matrix consisting of hydrogenated oil. For a separate set of capsules, microcapsules were produced and packed into HPMC capsules, without a lipid matrix. In parallel, an aqueous matrix, not microencapsulated, was dispensed into capsules. Finally, in another set of capsules, microcapsules were mixed with hydrogenated oil to form a slurry, which was then packed into capsules. It was expected that microencapsulation of the aqueous suspension would protect the outer capsule shell from degradation, by absorbing the water present in the aqueous matrix and retaining it in a gel. Surprisingly, microencapsulation alone was ineffective at preventing breakdown of the capsule shell. This was because the porous capsule shell progressively wicked moisture out of the microcapsule gels by capillary action. Similarly, the mixture of aqueous matrix with hydrogenated oil was ineffective for the preservation of capsule integrity, because the aqueous phases separated and the aqueous phase degraded the capsule. Surprisingly, the combination of hydrogel microcapsules in a lipid matrix synergistically conferred excellent physical stability in the capsule system.

The capsules filled with a powder were used as positive control, and capsules filled with an aqueous solution of water with 15% glycerol and 2% salt as negative control.

As shown in Table B, capsules filled with NaCl had 100% integrity for 24 hours. Capsules filled with the aqueous solution degraded in under 1 hour. Capsules filled with microcapsules were slightly more stable than with the aqueous solution, however the outer capsule shell degraded with 24 hours, with associated shrinkage and dehydration of the microcapsules, as water passed from the microcapsules into the capsule shell by wicking action. Capsules with microcapsules in the lipophilic matrix were unexpectedly more stable than capsules filled only with microcapsules or with an aqueous solution, for 24 hours.

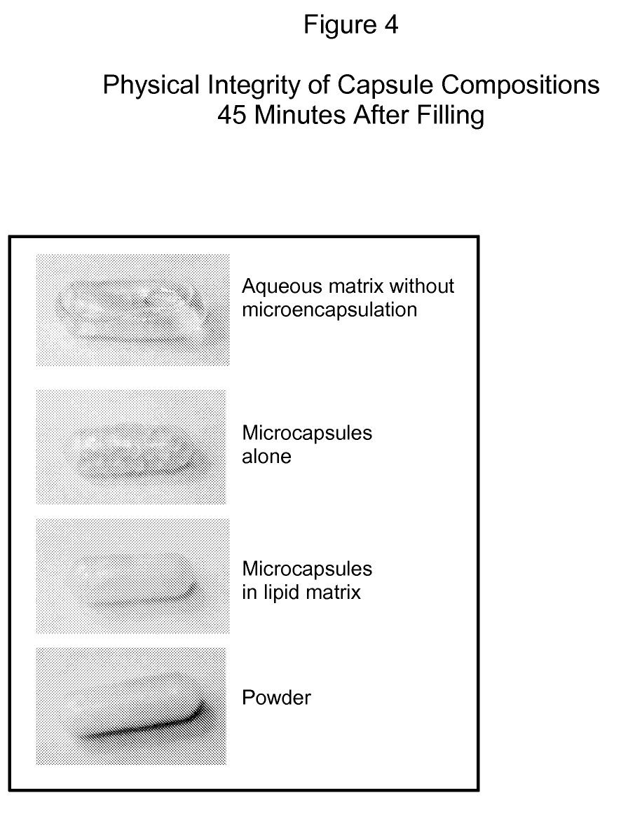

FIG. 4 shows representative photographs of capsules at 45 minutes post filling for each of the tested conditions.

### Example 3

### Capsule Integrity Assay

Example 3 examines capsule integrity for samples prepared as in Example 2, but immediately transferred to freezing conditions at −20° C. At 24 hours the samples were then transferred to room temperature.

The results are shown in Table C. Capsule conditions and scoring protocol were as described for Example 3. Table C shows that most of the capsules containing the aqueous cryoprotectant containing 15% glycerol and 0.2 M NaCl started to degrade in the freezer, and completely degraded once the capsules were transferred to room temperature. In contrast capsules containing microcapsules alone maintained integrity in the freezer, but immediately and completely degraded completely once the capsules were at room temperature. Microencapsulation alone does not confer stability on the outer capsule, because water can still pass from the microcapsules into the outer capsule by wicking action. Capsules with microcapsules in a lipophilic matrix were stable at −20° C., and retained stability at room temperature. Capsules filled with lipid alone, or powder alone, were completely stable, as a positive control.

TABLE C
Proportion of capsules with an intact shell following
storage under frozen and room temperature conditions.

| | | | After removal |
|---|---|---|---|
| | | After 48 | from freezer & |
| | | hours | 2 hours at |
| | | freezer | room |
| | Initial | storage | temperature |
| Capsule Design | % Intact | % Intact | % Intact |
| Microcapsules in lipid | 100% | 100% | 100% |
| matrix | | | |
| Microcapsules alone | 100% | 100% | 0% |
| Aqueous matrix alone | 100% | 40% | 0% |
| without microencapsulation | | | |
| Aqueous matrix, without | 100% | 60% | 0% |
| microencapsulation, with | | | |
| lipid matrix | | | |
| Lipid matrix alone | 100% | 100% | 100% |
| Powder | 100% | 100% | 100% |

### Example 4

### Unsatisfactory Recovery of Viable Fecal-Derived Bacteria Following Dehydration

Example 4 demonstrates a method for assessing the viability of a complex mixture of fecal derived bacteria.

The results demonstrate that fecal derived bacteria generally do not survive freeze drying.

A Freeze-Drying Buffer was prepared having the following ingredients:

(a) 0.3 g Tryptic Soy Broth

(b) 4 g sucrose

(c) 2 g Bovine Serum Albumin.

Ingredients (a), (b) and (c) were diluted in 40 mL of water and passed through a 0.2 μm filter.

Bacteria were extracted using the procedure of Example 1 and multiple aliquots were suspended in:

(1) 15% glycerol, 0.9% NaCl in water; or,

(2) the freeze-drying buffer

A first aliquot was plated for culture, and a second aliquot was frozen in dry ice. A third sample was freeze dried and transferred to −80° C.

Following overnight frozen storage, the resulting samples were recovered, resuspended in 0.9% NaCl and plated in serial dilution over Brain Heart Infusion agar plates. Plates were incubated anaerobically for 48 hours and colony forming units (CFUs) were counted.

Table E compares the number of CFUs between samples that were: (a) not subjected to freezing or freeze-drying, (b) freeze-dried samples, and (c) frozen samples.

Where the density of bacteria was so high as to form a continuous lawn; such results are indicated as “Lawn”. The results showed that no detectable fecal derived bacteria survived the freeze-drying process; while some bacteria survived freezing on dry ice. The bacteria not subjected to freezing or freeze-drying showed a great many CFUs indicating that there were a large number of viable bacteria.

TABLE E
Viability of Fecal-Derived Bacteria Subjected to Preservation
by Dehydration or Freezing in Aqueous Cryoprotectant

| Dilution | 1:100 | 1:1000 | 1:1000000 |
|---|---|---|---|
| No freezing | Lawn | 139 | None |
| Dehydration in | None | None | None |
| cryoprotectant | | | |
| Freezing in | 153 | 25 | None |
| aqueous | | | |
| cryoprotectant | | | |

### Example 5

### Preservation of Viable Fecal-Derived Bacteria in Capsules

Example 5 demonstrates that viable fecal-derived bacteria can be recovered from microcapsules.

Capsules and microcapsules containing fecal derived bacteria were prepared as in Example 1. Simulated intestinal fluid (SIF) was prepared according to established protocols of the U.S. Pharmacopeia Convention. See United States Pharmacopeia (USP 26).

Up to 90% of the capsule bodies of HPMC acid resistant capsules (size 0) were filled with fecal derived bacteria in microcapsules (containing that 15% glycerol and 1% sodium alginate), which were suspended in hydrogenated oil in a slurry. Other capsules were filled with fecal-derived bacteria in an aqueous buffer (not microencapsulated) that included 15% glycerol, and 0.2 M NaCl. Other capsules were filled with fecal-derived bacteria suspended in oil. Other capsules were filled with fecal-derived bacteria, which were lyophilized as in the preceding example.

The capsules were placed in sealed screw-cap plastic tubes which were then stored for one week at −20° C. Notably, the capsules which contained aqueous (no-microencapsulated) aqueous matrix mostly degraded during freezer storage. The capsules, or the contents of the capsules in the case of degraded capsules, were released into separate incubation tubes containing 30 mL of SIF. The tubes were agitated for 2 hours at room temperature, in order to allow the microcapsules to dissolve, and release their contents. The bacteria were recovered by centrifugation at 4000 rpm for 30 minutes and washed twice by resuspending the pellet with the 10 mL of isotonic buffer followed by centrifugation at 4000 rpm the for 30 minutes. The bacterial pellet was finally resuspended in 10 mL of PBS, pH=7.0.

Bacteria were stained with the Life Technologies Live/Dead bacterial viability assessment kit (BacLight™) to assess the viability of the bacteria. The BacLight™ viability kit utilizes a mixture of SYTO® 9 green-fluorescent nucleic acid stain and the red-fluorescent nucleic acid stain, propidium iodide. These stains differ both in their spectral characteristics and in their ability to penetrate healthy bacterial cells. The excitation/emission maxima for these dyes are 480/500 nm for SYTO® 9 and 490/635 nm for propidium iodide. When used alone, the SYTO® 9 generally labels all bacteria in a population—those with intact membranes and those with damaged membranes. In contrast, propidium iodide penetrates only bacteria with damaged membranes, causing a reduction in the SYTO® 9 stain fluorescence when both dyes are present.

To measure the relative ratio of live vs. dead bacteria 3 uL of the final suspension of stained bacteria were placed on a glass slide and observed under an inverted-fluorescence microscope (OLYMPUS IX83), which allows visualization of the samples in the screen of a computer. To obtain clear images with similar intensity, exposure parameters were defined for each filter. Images of different fields from the same sample were saved (for each field two pictures are generated, one for each filter/dye) and the number of dead/live bacteria of these was determined manually with the image editor software ImageJ (NIH) by using the function “Cell Counter”.

Table F represents the viable bacteria as percent total bacteria for each of the tested conditions. Forty-five percent (45%) of the total microencapsulated bacteria were viable for microencapsulated samples incubated −200 C for one week. The percent viable bacteria for samples incubated in an aqueous matrix without microencapsulation was 60%. The results demonstrated that the fecal derived bacteria were viable using capsules of microcapsules in a lipophilic matrix. This condition, unlike the non-microencapsulated matrix, produced capsules that were stable throughout the storage period. Few viable cells were recovered from the capsules prepared using lyophilization or lipid matrix alone, despite the physical stability of those capsules.

TABLE F
Viability of fecal-derived bacteria stored in capsules.

| | % viable |
|---|---|
| Capsule condition | bacteria |
| Microcapsules with lipid | 45 |
| matrix | |
| Aqueous matrix without | 60 |
| microencapsulation | |
| Freeze dried bacteria | 6 |
| Lipid matrix alone | 8 |

### Example 6

### Preservation of Anaerobic Bacteria in Capsules

In this example, representative non-spore forming anaerobic bacteria were preserved in stable capsules. The effects of oxygen, storage time and temperature were assessed. Preservation of bacterial viability was compared with a standard cryopreservation method.

A clinical isolate of Bacteroides fragilis, designated strain DH5042, was obtained from a patient and preserved as a glycerol stock.

The following steps were performed in an anaerobic glove box (Anaerobe Systems) in which the oxygen level was verified to be less than 30 parts per million.

The stock was streaked on a Brucella culture plate (Anaerobe Systems) to obtain a single isolated colony. The colony was inoculated into PRAS PY-Glucose broth and cultured anaerobically for 48 hours at 37° C.

The culture was dispensed into two tubes and centrifuged at 4,000 RPM in a clinical centrifuge to obtain Pellets A and B. Each of the pellets was suspended in a different sterile solution, at a concentration of 2% mass/volume. Pellet A was suspended in a solution containing 2% sodium alginate (mass/volume) in water (Suspension A). Pellet B was suspended in a solution containing 2% sodium alginate (mass/volume) and 15% glycerol (volume/volume) in water (Suspension B). The glycerol included in Suspension B served as a cryoprotectant for later freezing.

A 10-fold dilution series was prepared from Suspension B using sterile saline. 100 microliters from each diluted bacterial suspension was plated onto a Brucella plate for quantitative assessment of the density of colony forming units at baseline. This was performed to quantify viable bacteria at baseline, prior to any further manipulations. The culture plates were incubated at 37° C. anaerobically; 48 hours later. The cultures were subjected to analysis as described below.

Six aliquots of Suspension B were placed into sterile plastic tubes and immediately placed in a −80°C. freezer. This condition is called ‘Glycerol Stock’ and corresponds to the most widely used standard method for preserving bacteria in an aqueous frozen condition. For this experiment, it served as a positive control. Although this method is very reliable for a wide variety of bacteria, it is not compatible with the production of stable capsules for oral administration.

At this point, Suspensions A and B were split into aliquots. One aliquot of each was retained in the anaerobic environment. The other was placed in an aerobic environment (laboratory bench). The purpose of this was to evaluate the effect on bacterial viability of oxygen exposure during sample processing and storage.

Each of the four suspensions was separately microencapsulated using an extrusion method. Suspensions were placed into syringes and forced through a 23 gauge needle to form droplets, which were immediately immersed in a 0.1 M calcium chloride solution to polymerize the alginate. The resulting microcapsules were recovered by filtration, suspended in vegetable oil and packed into size 0 HPMC capsules (CapsCanada). Twelve capsules from each condition were prepared. The mass of microcapsules dispensed into each capsule was recorded. Capsules were hermetically sealed in groups of three in foil packages. Capsules produced from Suspension A were stored at 4° C. Capsules produced from Suspension B were stored at −80° C. The purpose of the foil sealed packets was to preserve the anaerobic environment (for capsules process in the anaerobic glove box) or to preserve the aerobic environment (for capsules processed in ambient air) during storage.

At specific time points post encapsulation, a set of capsules corresponding to each storage condition were placed into 10 mL sterile phosphate-buffered saline and rocked on a platform for 2 hours at room temperature. The capsules and microcapsules were solubilized, releasing the bacteria. The resulting liquid suspension was mixed well, diluted and plated for quantification of colony forming units as described in Step H below. In parallel, at each time point, a Glycerol Stock sample was analyzed for colony forming units as a positive control and comparator.

For analysis of CFUs, in an anaerobic glove box, the bacterial suspension to be analyzed was serially diluted using sterile saline. The resulting diluted suspensions were plated onto Brucella plates and the plates were incubated anaerobically for 48 hours at 37° C. Following incubation, a plate that exhibited between 30 and 300 discrete colonies was selected for counting. Colonies were counted and the CFU per gram bacterial mass input (CFU/g) was calculated using the dilution factor, the plated volume and the mass/volume concentration of bacteria in the initial suspension.

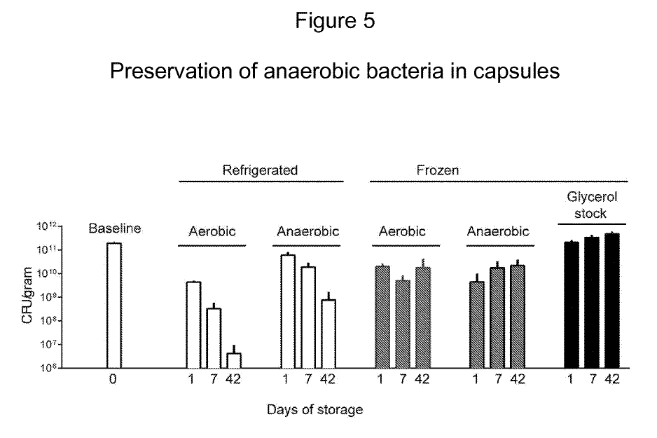

The results of the experiment are depicted in FIG. 5. These results demonstrated that:

a. A frozen glycerol stock preserved bacterial viability, as expected.

b. Viable bacteria were recovered from capsules prepared according to the present invention.

c. Bacteria preserved in capsules retained significant viability when stored at 4° C., demonstrating that the invention can be used to preserve anaerobic bacteria without the need for freezing. However, this required anaerobic conditions.

d. Bacteria preserved in capsules retained significant viability when stored at −80° C. This preservation was not sensitive to the presence of oxygen during processing.

Together, these results demonstrate that the invention can be used to prepare stable capsules for the oral delivery of viable anaerobic bacteria, without the need to dehydrate the bacteria.

### Example 7

### Bacteria in Enteric Capsules Protected from Simulated Stomach Conditions and Released in Simulated Colon Conditions

In this example, capsules containing bacteria were prepared according to the present invention were coated with an enteric targeting film. The resulting capsules were shown to effectively protect the bacteria from simulated gastric conditions and to release viable bacteria when placed in simulated colon conditions.

Bacteria of the E. coli strain BW26113 are obtained as a frozen stock and inoculated into 100 mL of LB Broth media and growth overnight at 37° C. aerobically with shaking.

The culture was dispensed into centrifuge tubes and centrifuged at 3,850 g for 5 minutes. The pellets were resuspended in a total of 40 mL of a solution of 2% sodium alginate. Serial dilutions of this sample were prepared in LB Broth and plated onto LB agar plates and incubated at 37° C. for 48 hours, to quantify colony forming units at baseline, prior to any further manipulation.

The bacterial suspension (with no microencapsulation performed) was mixed with vegetable oil and dispensed into three size 0 gelatin capsules (Capsuline). Each capsule contained about 450 microliters of the suspension and 450 microliters of oil. These capsules began degrading immediately, and were therefore placed into screw cap tubes to contain the leaking contents.

The remaining bacterial suspension was microencapsulated using an emulsion method. 50 mg of calcium carbonate powder was mixed well with the bacterial suspension. The 40 mL suspension was mixed at 250 RPM with 200 mL vegetable oil to create an emulsion. 80 microliters of glacial acetic acid was dissolved in 20 mL vegetable oil and the resulting solution was added to the emulsion. Stable microcapsules formed in the oil matrix.

The resulting slurry, containing microcapsules suspended in an oil matrix, was dispensed in equal amounts into six size 0 gelatin capsules (Capsuline), 900 microliters per capsule. All capsules were capped and the seams were sealed by with a 2.5% gelatin solution.

A suspension was prepared containing 303 mL water, 606.1 g Eudragit FS 30 D coating compound (Evonik) and 90.9 g PlasACRYL T20 plasticizer (Evonik). The suspension was atomized and applied to some of the capsules in a thin film, using a spray paint gun. The film was allowed to dry and the coating process was repeated three times. The three resulting capsules had an even coat of Eudragit polymer. The purpose of this coat was to protect the capsules from gastric conditions, as this polymer is solubilized in neutral solutions (e.g. intracolonic fluid) but not in acidic conditions (e.g. stomach fluid). The insulated inner contents of the capsule would thus be protected from stomach acid and also from stomach proteases. Three of the six capsules containing the microencapsulated bacteria were coated; the other three such capsules remained uncoated. The three capsules containing the non-microencapsulated bacteria could not be coated, as they had fallen apart.

Each capsule was placed in a tube containing 40 mL 0.1 M hydrochloric acid and gently agitated on a rocking platform for 30 minutes. The three coated capsules remaining intact during the incubation. For each capsule that had fallen apart, the broken capsule and capsule contents were recovered and placed into a tube containing 40 mL 0.1 M hydrochloric acid and gently agitated on a rocking platform for 30 minutes. The three uncoated capsules and the broken capsules completely released their contents; however microcapsules remained intact. (The depolymerization of the alginate depends on the presence of phosphate buffering ions, present in colonic fluid but not in gastric fluid.)

After the 30-minute incubation, intact capsules were transferred to three tubes containing 40 mL of simulated intestinal fluid (SIF) (water containing 0.1 M phosphate buffer, pH 7) and rocked for 2 hours at room temperature. For tubes containing broken capsules, contents (bacteria and microcapsules) were recovered by centrifugation, resuspended in 40 mL SIF and rocked for 2 hours at room temperature.

All capsules and microcapsules were fully dissolved during the SIF incubation. A small volume of SIF was sampled from each tube after gentle but thorough mixing. The bacterial suspensions to be analyzed were serially diluted using sterile saline. The resulting diluted suspensions were plated onto LB plates and the plates were incubated aerobically for 48 hours at 37° C. Following incubation, a plate that exhibited between 30 and 300 discrete colonies was selected for counting. Colonies were counted and the CFU per gram bacterial mass input (CFU/g) was calculated using the dilution factor, the plated volume and the mass/volume concentration of bacteria in the initial suspension.

The results are depicted in Table G. Viable bacteria were recovered from the coated capsules containing microcapsules in a lipid matrix. Recovery of bacteria from other capsules was reduced by 6 logs, demonstrated that capsules prepared according to the present invention are able to be combined with known coating methods to protect orally administered capsules from gastric digestion. Capsules prepared using aqueous suspensions of bacteria, according to previously described methods, were not compatible with such coating methods.

TABLE G
Viability of cultured bacteria following encapsulation
and exposure to simulated gastrointestinal conditions

| | Colony forming |
|---|---|
| | units recovered per |
| | mL of input |
| Capsule | bacterial |
| condition | suspension |
| Baseline (pre | 2.5 × 10¹³ |
| encapsulation) | |
| Microcapsules with | 1.5 × 10¹³ |
| lipid matrix and | |
| enteric coat | |
| Microcapsules with | 4.5 × 10⁶ |
| lipid matrix without | |
| enteric coat | |
| Aqueous matrix without | 8.2 × 10⁵ |
| microencapsulation | |

### Example 8

### Antibody in Capsules Protected from Stomach Conditions and Released In Colon Conditions

In this example, polyclonal antibody was loaded into capsules prepared according to the present invention. The resulting capsules effectively protected the antibody from simulated gastric conditions and release the antibody in simulated colonic fluid. These results were compared with previously described methods of delivering antibodies orally: as a drinkable liquid or as a lyophilized powder in capsules.

Human blood-derived polyclonal antibody was obtained (Sigma Aldrich Chemicals).

Three size 00 HPMC capsules (CapsCanada) were each filled with 9 mg antibody.

A solution was prepared containing 4% mass/volume antibody and 1% mass/volume sodium alginate. An aliquot of this solution was reserved for later steps. The remainder of the solution was microencapsulated using an extrusion method. Solution was placed into syringes and forced through a 23 gauge needle to form droplets, which were immediately immersed in a 0.1 M calcium chloride solution to polymerize the alginate. The resulting microcapsules were recovered by filtration, lightly dried by blotting, suspended in coconut oil and packed into size 00 HPMC capsules (CapsCanada). Each capsule received approximately 225 mg of microcapsules. Three capsules from each condition were prepared.

Capsules were capped and sealed with a 2.5% gelatin solution.

One capsule from each condition was set aside. The remaining capsules were coated with an enteric targeting film. A suspension was prepared containing 303 mL water, 606.1 g Eudragit FS 30 D coating compound (Evonik) and 90.9 g PlasACRYL T20 plasticizer (Evonik). The suspension was filtered, atomized and applied to some of the capsules in a thin film, using a spray paint gun. The film was allowed to dry and the coating process was repeated four times. The resulting capsules had an even coat of Eudragit polymer. The purpose of this coat was to protect the capsules from gastric conditions, as this polymer is solubilized in neutral solutions (e.g. intracolonic fluid) but not in acidic conditions (e.g. stomach fluid). The insulated inner contents of the capsule would thus be protected from stomach acid and also from stomach proteases.

A simulated gastric solution was prepared using 0.1 M hydrochloric acid and 0.32% purified pepsin (from porcine stomach; Sigma Aldrich) in water. Four 40 mL aliquots were made in conical tubes. The following antibody preparations were then added:

1. Enteric coated capsule filled with microencapsulated antibody in a lipid matrix.

2. Enteric coated capsule filled with lyophilized antibody.

3. 225 microliters of solution containing 4% antibody and 1% alginate (not microencapsulated nor in a capsule).

4. Capsule filled with 225 microliters of solution containing 4% antibody and 1% alginate (not microencapsulated).

The preparations above were rotated at 37 degrees Celsius for 1 hour. The capsule from condition 4 released the cargo solution within a few minutes.

For conditions 3 and 4, where antibody was released into the acidic solution, the solutions were passed through a 300 micron mesh filter and concentrated approximately 80-fold using centrifugal concentrators (Amicon/Millipore).

For the remaining conditions, capsules were moved to 40 mL aliquots of a 0.1 M phosphate buffer solution (pH 6.8) and rotated at 37 degrees Celsius for 3 hours. The capsules and microcapsules dissolved to the point where few particles of gelatin or alginate were overtly visible by eye.

The resulting solutions were passed through a 300 micron mesh filter and concentrated approximately 80-fold using centrifugal concentrators (Amicon/Millipore).

The samples were analyzed by standard SDS-PAGE gel electrophoresis using precast non-reducing gels (Novex) and a sample buffer containing SDS (Life Technologies). A volume was loaded which would correspond to approximately 125 ng of antibody, assuming full recovery.

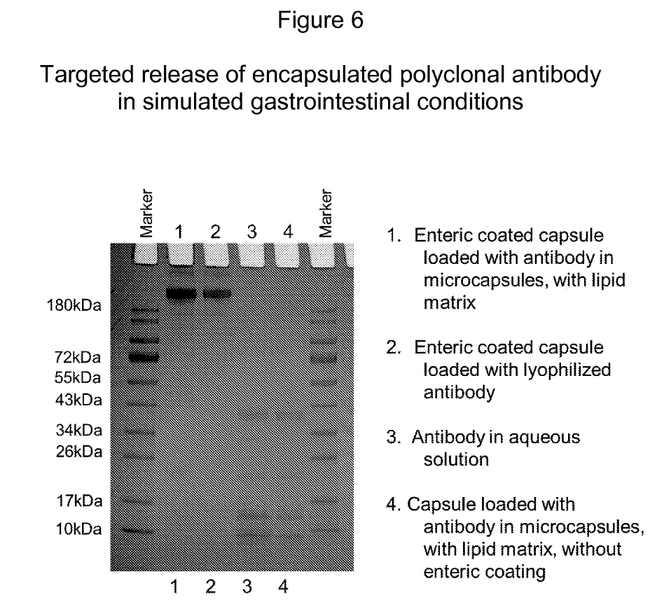

The results are illustrated in FIG. 6. The samples corresponding to conditions 1 and 2 resulted in a dominant, high-molecular-weight band corresponding to whole undigested IgG, indicating that capsules prepared according to the present invention could effectively protect IgG from simulated gastric conditions and then release IgG in simulated colonic fluid. Capsules packed with lyophilized IgG were also effective for this purpose; however, it is often desirable to continuously retain therapeutic IgG in an aqueous environment, which is not possible when utilizing that capsule design. When aqueous IgG solution was pipetted directly into capsules, without microencapsulation, enteric coating was impossible due to capsule instability, resulting in release of liquid IgG solution into the simulated gastric fluid. This was similar to the liquid IgG condition, simulating a situation where a patient would drink a liquid containing a therapeutic antibody in solution. Although the retention in an aqueous environment is achieved in both conditions, characteristic acid- and pepsin-mediated degradation of the IgG molecule was observed, evident by the absence of the high-molecular-weight IgG band and appearance of smaller bands corresponding to degradation products.

### Example 9

### Treatment of C. Difficile Infection in Male Golden Syrian Hamsters with Streptococcus Faecalis

Capsules containing Streptococcus faecalis are prepared as in Example 7, except that capsules for veterinary use (Torpac, N.J., USA) are used. Each capsule containing a homogenous mixture of microcapsules, wherein each microcapsule contains essentially homogenous Streptococcus faecalis.

On day 0, male golden Syrian hamsters are orally challenged by gavage with C. difficile strain. The capsules with S. faecalis are administered to the infected hamsters on day 7. Blood and stool are collected via retro-orbital bleeding.

Severity of C. difficile infection is assessed by monitoring for morbidity (weight loss) and mortality (death) typically associated with C. difficile infection in hamsters.

The results show that the capsules effectively treated C. difficile infections.

### Example 10

### Treatment of C. Difficile in Male Golden Syrian Hamsters with Bacillus Mesentericus.

Capsules containing Bacillus mesentericus are prepared as in Example 7, except that capsules for veterinary use (Torpac, N.J., USA) are used.

On day 0, male golden Syrian hamsters are orally challenge by gavage with C. difficile. The capsules with B. mesentericus are administered to the infected hamsters on day 7. Blood and stool are collected on days via retro-orbital bleeding.

Severity of C. difficile infection is assessed by monitoring for morbidity (weight loss) and mortality (death) typically associated with C. difficile infection in hamsters.

The results show that the capsules effectively treat C. difficile infections.

### Example 11

### Treatment of C. difficile Infection in Male Golden Syrian Hamsters with Streptococcus Faecalis and Bacillus Mesentericus

Capsules containing Bacillus mesentericus and Streptococcus faecalis are prepared as in Example 7 and FIG. 2b, except that capsules for veterinary use (Torpac, N.J., USA) are used.

Fifty-percent of the microcapsules contain essentially homogenous B. mesentericus. Fifty-percent of the microcapsules contain essentially homogenous S. faecalis.

On day 0, male golden Syrian hamsters are orally challenge by gavage with C. difficile strain 630 (500 CFU). The capsules with B. mesentericus are administered to the infected hamsters. Blood and stool are collected via retro-orbital bleeding.

Severity of C. difficile infection is assessed by monitoring for morbidity (weight loss) and mortality (death) typically associated with C. difficile infection in hamsters.

The results show that the capsules effectively treat C. difficile infections.

### Example 12

### Treatment of C. difficile Infection in Male Golden Syrian Hamsters with Capsules of C. scindens, Barnesiella intestihominis, Pseudoflavonifractor Capillosus and Blautia Hansenii.

Capsules are prepared, as in Example 7 and FIG. 2b, except that capsules for veterinary use (Torpac, N.J., USA) are used, containing a heterogeneous populations of microcapsules. About twenty-five percent of the microcapsules contain essentially homogenous C. scindens. About twenty-five percent of the microcapsules contain essentially homogenous Barnesiella intestihominis. About twenty-five percent of the microcapsules contain essentially homogenous Pseudoflavonifractor capillosus. About twenty-five percent of the microcapsules contain essentially homogenous Blautia hansenii.

On day 0, male golden Syrian hamsters are orally challenged by gavage with C. difficile. The capsules are administered to the infected hamsters on day 7. Blood and stool are collected via retro-orbital bleeding.

Severity of C. difficile infection is assessed by monitoring for morbidity (weight loss) and mortality (death) typically associated with C. difficile infection in hamsters.

The results show that the capsules effectively treat C. difficile infections.

### Example 13

### Prevention of C. difficile Infection in Male Golden Syrian Hamsters with Capsules of C. Scindens, Barnesiella Intestihominis, Pseudoflavonifractor Capillosus and Blautia Hansenii

Capsules are prepared as in Example 7, except that capsules for veterinary use (Torpac, N.J., USA) are used.

On day 0, Male golden Syrian hamsters are administered capsules. On day 7, the hamsters are orally challenge by gavage with C. difficile. Weight, Blood and stool are collected on days via retro-orbital bleeding.

The capsules prevent C. difficile infection and protect against morbidity (weight loss) and mortality (death) associated with C. difficile.

### Example 14

### Prevention of C. difficile Infection in Male Golden Syrian Hamsters with Capsules of C. Scindens

Capsules containing microcapsules with vegetative C. scindens cells are prepared as in Example 7, except that capsules for veterinary use (Torpac, N.J., USA) are used.

On days 0 to 7, male golden Syrian hamsters are administered penicillin by water. Prior to the experiment, hamsters are not colonized with C. scindens as determined by a specific PCR assay targeting the 16S rDNA genomic sequence in bacterial DNA prepared from stool samples. Male golden Syrian hamsters are administered capsules on day 8. On day 10, the hamsters are orally challenge by gavage with C. difficile strain. Blood and stool are collected via retro-orbital bleeding.

Severity of C. difficile infection is assessed by monitoring for morbidity (weight loss) and mortality (death) typically associated with C. difficile infection in hamsters.

The capsule administration results in colonization of the hamsters with C. scindens, as determined by a specific PCR assay targeting the 16S rDNA genomic sequence in bacterial DNA prepared from stool samples. This demonstrates the ability of the present invention to stably transfer viable bacteria to the gastrointestinal system of an animal.

The capsule administration prevents C. difficile infection and protect against morbidity (weight loss) associated with C. difficile.

### Example 15

### Prevention of C. difficile Infection in Male Golden Syrian Hamsters with Capsules of Barnesiella Intestihominis

Capsules containing microcapsules with Barnesiella intestihominis are prepared as in Example 7, except that capsules for veterinary use (Torpac, N.J., USA) are used.

On days 0 to 7, male golden Syrian hamsters are administered penicillin by water. Hamsters are administered capsules. On day 10, the hamsters are orally challenge by gavage with C. difficile. Weight, Blood and stool are collected via retro-orbital bleeding.

The capsules prevent C. difficile infection and protect against morbidity (weight loss) and mortality (death) associated with C. difficile infection.

### Example 16

### Prevention of C. difficile Infection in Male Golden Syrian Hamsters with Capsules of Pseudoflavonifractor Capillosus

Capsules containing microcapsules with Pseudoflavonifractor capillosus are prepared as in Example 7 except that capsules for veterinary use (Torpac, N.J., USA) are used.

On days 0 to 7, male golden Syrian hamsters are administered penicillin by water. On days 8, male golden Syrian hamsters are administered capsules twice a day. On day 10, the hamsters are orally challenge by gavage with C. difficile. Weight, Blood and stool are collected on days via retro-orbital bleeding.

The capsules prevent C. difficile infection and protect against morbidity (weight loss) and mortality (death) associated with C. difficile.

### Example 17

### Prevention of C. difficile Infection in Male Golden Syrian Hamsters with Capsules of Blautia Hansenii

Capsules containing microcapsules with Blautia hansenii are prepared as in Example 7, except that capsules for veterinary use (Torpac, N.J., USA) are used.

On days 0 to 7, male golden Syrian hamsters are administered penicillin by water. On days 8, male golden Syrian hamsters are administered capsules twice a day. On day 10, the hamsters are orally challenge by gavage with C. difficile strain. Weight, Blood and stool are collected on via retro-orbital bleeding.

The capsules prevent C. difficile infection and protect against morbidity (weight loss) and mortality (death) associated with C. difficile.

### Example 18

### Prevention of C. difficile Infection in Male Golden Syrian Hamsters with Capsules of Acidaminococcus intestinalis

Capsules containing microcapsules with Acidaminococcus intestinalis are prepared as in Example 7, except that capsules for veterinary use (Torpac, N.J., USA) are used.

On days 0 to 7, male golden Syrian hamsters are administered penicillin by water. On days 8, the hamsters are administered capsules twice a day. On day 10, the hamsters are orally challenge by gavage with C. difficile strain 630 (500 CFU). Weight, Blood and stool are collected on days 0, 1, 3, 5, 10 14 and 21 via retro-orbital bleeding.

The capsules prevent C. difficile infection and protect against morbidity (weight loss) and mortality (death) associated with C. difficile.

### Example 19

### Prevention of C. difficile Infection in Male Golden Syrian Hamsters with Capsules Comprising a Heterogeneous Mixture of Bacteria in Each Microcapsule

Microcapsules are prepared as in Example 7, except that capsules for veterinary use (Torpac, N.J., USA) are used, with a mixture of the following bacteria in each microcapsule: Acidaminococcus intestinalis; Bacteroides ovatus; Bifidobacterium adolescentis; Bifidobacterium longum; Clostridium cocleatum; Blautia product; Collinsella aerofaciens; Dorea longicatena; Escherichia coli; Eubacterium desmolans; Eubacterium eligens; Eubacterium limosum; Eubacterium rectale; Eubacterium ventriosum; Faecalibacterium prausnitzii; Lachnospira pectinoshiza; Lactobacillus casei/paracasei; Lactobacillus casei; Ruminococcus torques, Parabacteroides distasonis; Raoultella sp.; Roseburia faecalis; Roseburia intestinalis; and Ruminococcus obeum.

On days 0 to 7, male golden Syrian hamsters are administered penicillin by water. Syrian hamsters are administered capsules. On day 10, the hamsters are orally challenged by gavage with C. difficile strain 630 (500 CFU). Weight, Blood and stool are collected on days 0, 1, 3, 5, 10 14 and 21 via retro-orbital bleeding.

The capsules prevent C. difficile infection and protect against morbidity (weight loss) and mortality (death) associated with C. difficile.

### Example 20

### Prevention of C. difficile Infection in Male Golden Syrian Hamsters with Capsules Comprised of a Heterogeneous Mixture of Microcapsules

Capsules are prepared as in Example 7 and FIG. 2b, except that capsules for veterinary use (Torpac, N.J., USA) are used, containing a heterogeneous mixture of microcapsules. Different population of microcapsules containing essentially one species of the following bacteria per microcapsule are prepared: Acidaminococcus intestinalis, Bacteroides ovatus, Bifidobacterium adolescentis, Bifidobacterium longum, Clostridium cocleatum, Blautia product, Collinsella aerofaciens, Dorea longicatena, Escherichia coli ,Eubacterium desmolans, Eubacterium eligens, Eubacterium limosum, Eubacterium rectale (four different strains), Eubacterium ventriosum, Faecalibacterium prausnitzii, Lachnospira pectinoshiza, Lactobacillus casei/paracasei, Lactobacillus casei, Ruminococcus torques (two different strains), Parabacteroides distasonis, Raoultella sp., Roseburia faecalis, Roseburia intestinalis, Ruminococcus obeum (two different strains), C. scindens, Barnesiella intestihominis, Pseudoflavonifractor capillosus or Blautia hansenii.

Each therapeutic capsule contains all of the above bacteria.

On days 0 to 7, male golden Syrian hamsters are administered penicillin by water. Male golden Syrian hamsters are administered capsules twice a day. On day 10, the hamsters are orally challenge by gavage with C. difficile. Weight, Blood and stool are collected on days 0, 1, 3, 5, 10 14 and 21 via retro-orbital bleeding.

The capsules prevent C. difficile infection and protect against morbidity (weight loss) and mortality (death) associated with C. difficile.

### Example 21

### Prevention of C. difficile Infection in Male Golden Syrian Hamsters with Capsules of C. scindens, Barnesiella intestihominis, Pseudoflavonifractor capillosus and Blautia hansenii and Faecalibacterium prausnitzii

A capsule comprising different loads of bacteria are prepared as in Example 7 and FIG. 2b, except that capsules for veterinary use (Torpac, N.J., USA) are used.

A single capsule will contain microcapsules with different loads. Clostridium scindens (ATCC 35704) providing infection resistance against C. difficile has been reported by Buffie et el. (Nature. 2015 Jan 8;517(7533):205-8. Precision microbiome reconstitution restores bile acid mediated resistance to Clostridium difficile.) Adoptive oral capsule-mediated transfer of C. scindens or a capsule containing equal numbers of C. scindens, Barnesiella intestihominis, Pseudoflavonifractor capillosus (ATCC 29799), Blautia hansenii (ATCC 27752) and Faecalibacterium prausnitzii (ATCC 27766).

Capsules containing C. scindens, Barnesiella intestihominis, Pseudoflavonifractor capillosus, Faecalibacterium prausnitzii and Blautia hansenii are given to antibiotic-treated mice lead to protection against morbidity (weight loss) and mortality (death) in a prevention scenario against Clostridium difficile. All bacteria are grown under anaerobic conditions in reduced Brain-Heart Infusion media supplemented with yeast extract and cysteine except for B. intestihominis, which is grown in liquid Wilkins-Chalgren media, and resuspended in anaerobic PBS prior to capsule preparation. A solution is produced containing a pooled population of bacteria, consisting of ten million CFU of each bacterial strain; 1.5% sodium alginate in solution; and an insoluble form of calcium, as a suspension, at a concentration of about 1% mass/volume. The resulting liquid is added to vegetable oil in a ratio of about 1:2 alginate solution to oil. The two solutions are mixed using a magnetic stir bar at 1000 RPM to produce a fine emulsion. Acetic acid, dissolved in vegetable oil, is added to the system. The acetic acid lowers the pH of the oil phase of the emulsion slightly, releasing the calcium ions from the calcium carbonate complex; the calcium ions are then available to bind to the alginate chains, triggering the polymerization and forming discrete microcapsules of bacteria-laden aqueous gel in a lipophilic matrix of vegetable oil. Resulting microcapsules are recovered by filtration, washed with a calcium chloride solution (0.1 molar concentration), lightly blotted and resuspended in liquefied vegetable oil. The resulting slurry is packed into capsules for veterinary use (Torpac, N.J.), which are then coated with an enteric coating according to described methods (Torpac.com).

On days 0 to 7, male golden Syrian hamsters are administered penicillin by water. Male golden Syrian hamsters are administered capsules. On day 10, the hamsters are orally challenge by gavage with C. difficile. Weight, Blood and stool are collected on days 0, 1, 3, 5, 10 14 and 21 via retro-orbital bleeding.

The capsules prevent C. difficile infection and protect against morbidity (weight loss) and mortality (death) associated with C. difficile.

### Example 25

### Prevention of C. difficile Infection in Mice with Capsules of C. scindens, Barnesiella intestihominis, Pseudoflavonifractor capillosus and Blautia Hansenii

A capsule comprising different loads of bacteria are prepared as in Example 21.

C57BL/6 mice (n=12) are infected with C. difficile via spore-mediated transmission and are treated with clindamycin for 7 days such as to induce a highly contagious “supershedder” stage as described in patent application WO 2013171515 A1. Intestinal dysbiosis is confirmed by 16S rRNA gene sequence profiling of bacterial DNA isolated from fresh fecal pellets (Shannon Diversity Index-SDI). Control groups include healthy/naive C57BL/6 mice (n=12) and clindamycin-treated C57BL/6 mice (n=12). Once a supershedder phenotype is established, a single treatment via oral gavage of C. scindens, a bacterial load consisting of several capsules, containing a total of 10⁹ c.f.u. of each strain C. scindens, Barnesiella intestihominis, Pseudoflavonifractor capillosus and Blautia hansenii, or vehicle, is be administered to 4 mice from each cohort mentioned above. Fecal pellets are collected daily for 16S rDNA sequencing and C. difficile shedding is monitored daily. Suppression of a supershedder state and an increase in bacterial diversity is indicative of a successful treatment. Protection in animals is observed following treatment with capsules prepared according to the present invention, as the capsule allows for the safe passage of the cargo through stomach acid, small intestinal enzymes, straight to the colon where the infection is occurring.

### Example 26

### Prevention of C. Difficile Infection in Mice with Capsules of C. scindens, Barnesiella Intestihominis, Pseudoflavonifractor Capillosus and Blautia Hansenii

A capsule comprising different loads of bacteria are prepared as in Example 7, except that capsules for veterinary use (Torpac, N.J., USA) are used.

Antibiotic-treated C57BL/6 mice (n=16) are challenged with 10⁶ spores of C. difficile UK1 (027/B1/NAP1) to establish a primary C. difficile infection (“CDI”) Control groups include healthy/naive C57BL/6 mice (n=16) and antibiotic-treated C57BL/6 mice (n=16). At day 3-5 post challenge when CDI is established, a single treatment via oral gavage of C. scindens, a bacterial suspension in one capsule containing C. scindens, Barnesiella intestihominis, Pseudoflavonifractor capillosus and Blautia hansenii, or PBS is administered to 4 mice from each cohort, along with vancomycin. The capsule allows for the safe passage of the cargo through stomach acid, small intestinal enzymes, directly to the colon where the infection is occurring. Mice are observed daily for signs of diarrhea and weights measured. Fecal pellets are collected daily for 16S rDNA sequencing. Protection in animals treated with capsules bearing bacteria is observed, as indicated by an increase in bacterial diversity, resolution of diarrhea and weight gain indicating successful treatment.

### Example 27

### Prevention of C. difficile Infection with a Complex Mixture of Bacteria

Capsules are prepared as in Example 26, containing microcapsules with Acidaminococcus intestinalis, Bacteroides ovatus, Bifidobacterium adolescentis, Bifidobacterium longum, Clostridium cocleatum, Blautia product, Collinsella aerofaciens, Dorea longicatena, Escherichia coli ,Eubacterium desmolans, Eubacterium eligens, Eubacterium limosum, Eubacterium rectale, Eubacterium ventriosum, Faecalibacterium prausnitzii, Lachnospira pectinoshiza, Lactobacillus casei/paracasei, Lactobacillus casei, Ruminococcus torques, Parabacteroides distasonis, Raoultella sp., Roseburia faecalis, Roseburia intestinalis, and Ruminococcus obeum, are made and used to prevent C. difficile infection as in Example 27 to protect against morbidity (weight loss) and mortality (death) against Clostridium difficile. Protection in animals treated with capsules bearing bacteria is observed, as the capsule allows for the safe passage of the cargo through stomach acid and small intestinal enzymes, straight to the colon where the infection is occurring.

### Example 28

### Delivery to the Cecum of Monoclonal Antibodies Targeting C. difficile Toxins

In one example capsules contain monoclonal antibodies CDA1 and MDX-1388. See: Infection for prevention of C. difficile. (Infect Immun. 2006 Nov;74(11):6339-47, Human monoclonal antibodies directed against toxins A and B prevent Clostridium difficile-induced mortality in hamsters; see also: Antibodies against Clostridium difficile toxins and uses thereof, U.S. Pat. No. 7625559 B2.) CDA1 and MDX-1388 sequences encoding the heavy and light chain variable regions are synthesized from published DNA sequences and cloned into vectors pCON-gammal and pCON-kappa (Antibodies against Clostridium difficile toxins and uses thereof, U.S. Pat. No. 7,625,559 B2). Full-length IgG1, κ monoclonal antibodies are expressed and purified from stably transfected CHO-K1SV cells and purified to >95% homogeneity by protein A chromatography (Clin Vaccine Immunol. 2013 Mar;20(3):377-90. A mixture of functionally oligoclonal humanized monoclonal antibodies that neutralize Clostridium difficile TcdA and TcdB with high levels of in vitro potency shows in vivo protection in a hamster infection model.)

Capsules containing CDA1 and MDX-1388 are formed by the emulsion method, as in Example 7.

Torpac 0.13 ml capsules are filled to 90% of the capsule volume with slurry of microcapsules in hydrogenated oil. Male golden Syrian hamsters are given a total of four doses of antibody for 4 days (days -3, -2, -1, and 0). CDA1 and MDX-1388 are administered at a dose of 50 mg/kg/day (total 100 mg/kg/day) either orally via capsule or via i.p. injection. Blood is collected at days 0, 1, 3, and 5 via retro-orbital bleeding to monitor levels of antibody circulating in the blood and shed in the stool.

Cecum contents are dissected on day 5. Intact (undigested, native) antibody in cecum content is detected by ELISA and Western blotting. Intact (undigested, native) antibody is detected within the cecum content of treated mice.

### Example 29

### Delivery of Anti-Checkpoint Inhibitor Antibody to the Colon

In some embodiments capsule-based delivery of monoclonal antibodies against PD-L1/anti-PD1 is evaluated. Anti-PD1 antibodies are produced as described. See U.S. Pat. No. 8,008,449 B2.

Capsules containing anti-PD1 antibodies are formed by the emulsion method as described in Example 7 and FIG. 2c. Microcapsules containing individual monoclonal antibodies are prepared separately then mixed together with oil to form a slurry.

0.13 mL capsules (Torpac.com) are filled to 90% of the capsule volume with slurry of microcapsules in oil. Capsules are coated with a film for enteric targeting as described by the manufacturer (Torpac.com).

Mice are given oral capsules daily for 4 days, with anti-PD-L1 antibody at 10 mg/kg dose, or intravenous anti-PD-L1 antibody daily for 4 days at the same dose. Colonic content is recovered by dissection in day 5.

Antibody levels in the colon contents are quantified by ELISA.

Anti-PD-L1 antibody is detected in the colon contents of capsule-treated mice. Lower levels are observed in the colon contents from intravenously-injected mice.

### Example 30

### Delivery of a Phage Lysin Protein to the Colon

In one example, single components of bacteriophage such as bacteriophage lytic enzymes, or endolysins, expressed in E.coli, are delivered to the colon using the capsule described herein. Endolysins or lysins are highly evolved molecules produced by bacteriophage to digest the bacterial cell wall for bacteriophage progeny release.

A putative amidase lysin, PlyCD-174, identified from the prophage genome of multi-drug resistant C. difficile strain CD630, is expressed in E.coli using His-tag expression system (Antimicrob Agents Chemother. 2015 Using a novel lysin to help control Clostridium difficile infections). 400 μg of PlyCD1-174, the enzymatic portion of the lysine, is packed in a capsule.

Capsules comprising PlyCD-174 are formed by the emulsion method as described in Example 7 and FIG. 2c.

0.13 mL capsules (Torpac.com) are filled to 90% of the capsule volume with slurry of microcapsules in oil. Capsules are coated with a film for enteric targeting as described by the manufacturer (Torpac.com).

Oral capsule formulations of PlyCD1-174, or liquid formulation of PlyCD1-174 or PB (vehicle alone) are given to mice orally over 6 days (0.1 mg/kg daily). All mice are followed for 7 days. Colon contents are collected by dissection on day 7. Levels of PlyCD1-174 in the colon are analyzed by ELISA. The capsule formulation of PlyCD1-174 results in higher detectable levels than in the liquid administration group.

1. A therapeutic capsule for the oral administration of a biopharmaceutical to the gastrointestinal system comprising a capsule shell enveloping a lipophilic matrix permeated with microcapsules, wherein each microcapsule comprises a hydrophilic matrix formed from an aqueous medium, stabilized into a discrete structure by a colloidal polymer, and contains the biopharmaceutical.

2. The capsule of claim 1, wherein the biopharmaceutical is selected from the group consisting of bacteria, proteins, antibiotics, antibodies, enzymes, viruses, viral particles, phages, nutrients, and lipophobic drugs of combinations thereof.

3. The capsule of claim 1, wherein the biopharmaceutical comprises bacteria.

4. The capsule of claim 3, wherein the bacteria is selected from the group consisting of C. scindens, Barnesiella intestihominis, Pseudoflavonifractor capillosus, Faecalibacterium prausnitzii and Blautia hansenii or any combination thereof.

5. The capsule of claim 1, wherein the biopharmaceutical comprises proteins.

6. The capsule of claim 1, wherein the biopharmaceutical comprises antibiotics.

7. The capsule of claim 1, wherein the biopharmaceutical comprises antibodies.

8. The capsule of claim 1, wherein the biopharmaceutical comprises enzymes.

9. The capsule of claim 1, wherein the biopharmaceutical comprises viruses.

10. The capsule of claim 1, wherein the biopharmaceutical comprises viral particles.

11. The capsule of claim 1, wherein the biopharmaceutical comprises phages.

12. The capsule of claim 1, wherein the biopharmaceutical comprises nutrients.

13. The capsule of claim 1, wherein the biopharmaceutical comprises lipophobic drugs.

14. The capsule of claim 1, wherein the biopharmaceutical requires an aqueous environment to maintain activity.

15. The capsule of claim 1, wherein the capsule shell is made of material that comprises a polymer selected from the group consisting of gelatin and hydroxypropyl methylcellulose.

16. The capsule of claim 1, wherein the lipophilic matrix comprises a digestible oil.

17. The capsule of claim 16, wherein the digestible oil is selected from the group consisting of a hydrogenated oil, coconut oil, soybean oil, corn oil and canola oil.

18. The capsule of claim 1, wherein colloidal polymer comprises a hydrocolloid polymer.

19. The capsule of claim 18, wherein the hydrocolloid polymer comprises an alginate polymer.

20. The capsule of claim 1, wherein the colloidal polymer comprises an amphipathic polymer.

21. The capsule of claim 1, wherein the colloidal polymer comprises casein.

22. The capsule of claim 1, wherein the colloidal polymer comprises a protein.

23. The capsule of claim 1, wherein the aqueous solution comprises a cryoprotectant.

24. The capsule of claim 1, wherein the cryoprotectant comprises glycerol.

25. The capsule of claim 1, wherein the cryoprotectant comprises PEG 200.

26. The capsule of claim 1, wherein the bacteria comprises bacteria isolated from fecal matter.

27. The capsule of claim 1, wherein the biopharmaceutical comprises a complex mixture of cultured bacteria.

28. A method of treating disease in a patient by administering the capsule of claim 1.

29. The method of claim 28, wherein the patient is a human patient.

30. The method of claim 28, wherein the patient is an animal patient.

31. The method of claim 28, wherein the injury is a Clostridium difficile infection of the gastrointestinal system.

32. A therapeutic capsule for the oral administration of a biopharmaceutical to the gastrointestinal system comprising a capsule shell enveloping a lipophilic matrix permeated with discrete microcapsules, wherein each microcapsule comprises a hydrophilic matrix formed from an internal phase comprising an aqueous medium, stabilized into a discrete structure by a colloidal polymer, and contains bacteria.

33. The capsule of claim 32, wherein the bacteria comprises a complex mixture isolated from fecal matter.

34. The capsule of claim 32, wherein the bacteria is selected from the group consisting of C. scindens, Barnesiella intestihominis, Pseudoflavonifractor capillosus, Faecalibacterium prausnitzii, and Blautia hansenii or any combination thereof.

35. The capsule of claim 32, wherein the bacteria comprises C. scindens.

36. The capsule of claim 32, wherein the bacteria Comprises Barnesiella intestihominis.

37. The capsule of claim 32, wherein the bacteria comprises Pseudoflavonifractor capillosus.

38. The capsule of claim 32, wherein the bacteria comprises Faecalibacterium prausnitzii.

39. The capsule of claim 32, wherein the bacteria comprises Blautia hansenii.

40. The capsule of claim 32, wherein the capsule shell comprises a polymer selected from the group consisting of gelatin and hydroxypropyl methylcellulose.

41. The capsule of claim 32, wherein the lipophilic matrix comprises a digestible oil.

42. The capsule of claim 41, wherein the digestible oil is selected from the group consisting of a hydrogenated oil, coconut oil, soybean oil, corn oil and canola oil.

43. The capsule of claim 32, wherein colloidal polymer comprises a hydrocolloid polymer.

44. The capsule of claim 43, wherein the hydrocolloid polymer comprises an alginate polymer.

45. The capsule of claim 32, wherein the colloidal polymer comprises an amphipathic polymer.

46. The capsule of claim 32, wherein the colloidal polymer comprises casein.

47. The capsule of claim 32, wherein the colloidal polymer comprises a protein.

48. The capsule of claim 32, wherein the aqueous solution comprises a cryoprotectant.

49. The capsule of claim 48, wherein the cryoprotectant is selected from the group consisting of glycerol and PEG 200.

50. The capsule of claim 32, wherein the bacteria comprises bacteria isolated from fecal matter.

51. The capsule of claim 32, wherein the complex mixture of mixture of bacteria comprises a cultured bacteria]

52. A method of treating an injury to the microbiota in a patient by administering the capsule of claim 32.

53. The method of claim 52, wherein the patient is a human patient.

54. The method of claim 52, wherein the patient is an animal patient.

55. The method of claim 52, wherein the injury is a Clostridium difficile infection of the gastrointestinal system.

56. A process for making a therapeutic capsule for the oral administration of a biopharmaceutical to the gastrointestinal system comprising the steps of:
a. suspending the biopharmaceutical in an isotonic solution containing a colloidal polymer;
b. forming microspheres comprising the colloidal polymer, wherein the polymer forms an internal phase that entraps the biopharmaceutical;
c. recovering and drying the microspheres;
d. suspending the microspheres in a lipophilic matrix to form a slurry; and,
e. packing a capsule with the slurry,
f. wherein the colloidal polymer is selected from the group consisting of hydrocolloid colloidal polymers and amphiphilic colloidal polymers.

57. The process of claim 56, wherein the lipophilic matrix comprises a digestible oil.

58. The process of claim 56, wherein the digestible oil is selected from the group consisting of a hydrogenated oil, coconut oil, soybean oil, corn oil and canola oil.

59. The process of claim 56, wherein colloidal polymer comprises a hydrocolloid polymer.

60. The process of claim 59, wherein the hydrocolloid polymer comprises an alginate polymer.

61. The process of claim 56, wherein the colloidal polymer comprises an amphiphilic polymer.

62. The process of claim 56, wherein the colloidal polymer comprises casein.

63. The process of claim 42, wherein the colloidal polymer comprises a protein.

64. The method of claim 28, wherein the disease is an ailment of: the alimentary tract and metabolism; bile and liver disorders; dysbiosis of the gastrointestinal tract; growth and/or colonization of the gastrointestinal tract by a pathogenic bacterium; reducing growth and/or colonization of the gastrointestinal tract by a pathogenic bacterium; reduction of one or more non-pathogenic bacteria in the gastrointestinal tract; ailments of the alimentary tract and acid-related disorders; nausea; constipation; diarrhea; intestinal inflammation and infections; obesity; diet related disorders; blood and blood forming organs; the cardiovascular system; hypertension or high concentrations of lipid; dermatological systems; genito-urinary system; adrenal pituitary; hypothalamic disorders; pancreatic disorders calcium homeostasis; the immune system; musculoskeletal system and bone disease, musculonervous system diseases, the respiratory system including obstructive airway diseases; cough and cold; other respiratory system diseases; allergies; or combinations thereof.
